(19) United States
(12) Patent Application Publication
(10) Pub. No.: US 2022/0168322 A1
(43) Pub. Date: Jun. 2, 2022

(54) NOVEL STAT3 INHIBITORS IDENTIFIED BY STRUCTURE-BASED VIRTUAL SCREENING INCORPORATING SH2 DOMAIN FLEXIBILITY

(71) Applicants: Board of Regents, The University of Texas System, Austin, TX (US); The Methodist Hospital dba Houston Methodist Hospital, Houston, TX (US)

(72) Inventors: David John TWEARDY, Houston, TX (US); Ren KONG, Changzhou City (CN); Uddalak BHARADWAJ, Houston, TX (US); Moses Makokha KASEMBELI, Houston, TX (US); Stephen T. C. WONG, Missouri City, TX (US)

(73) Assignees: Board of Regents, The University of Texas System, Austin, TX (US); The Methodist Hospital dba Houston Methodist Hospital, Houston, TX (US)

(21) Appl. No.: 17/261,536

(22) PCT Filed: Jul. 19, 2019
(86) PCT No.: PCT/US19/42663

§ 371 Date: Jan. 19, 2021

Related U.S. Application Data
(60) Provisional application No. 62/701,001, filed on Jul. 20, 2018.

(51) Int. Cl. A61K 31/635; A61K 45/06; A61K 31/122; A61K 31/167; A61K 31/427; A61K 31/496; A61K 31/426; A61K 31/454; A61P 35/00

(52) U.S. Cl. CPC A61K 31/635; A61K 45/06; A61K 31/122; A61K 31/167; A61P 35/00; A61K 31/496; A61K 31/426; A61K 31/454; A61K 31/427

(57) ABSTRACT

In one aspect, the present disclosure provides methods of inhibiting STAT3 in a cell comprising contacting the cell with a compound of the formula: (I) wherein the variables are as defined herein. In another aspect, the present disclosure provides methods of using of the compounds disclosed herein for the treatment of cancer.

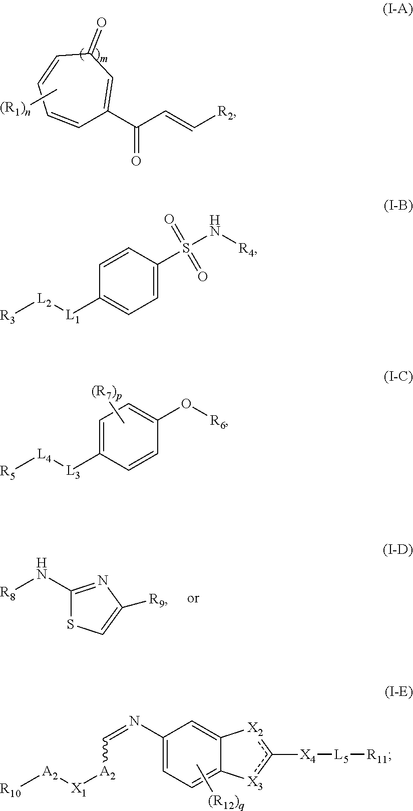

The present application is a national phase application under 35 U.S.C. § 371 of International Application No. PCT/US2019/042663, filed Jul. 19, 2019, which claims the priority benefit of U.S. provisional application No. 62/701,001, filed Jul. 20, 2018, the entire contents of each of which are incorporated herein by reference.

### BACKGROUND

### 1. Field

The present invention relates generally to the treatment of cell proliferative diseases such as cancer. More particularly, it concerns methods of inhibiting STAT3 using small-molecule STAT3 inhibitors as well as methods for the treatment of cell proliferative diseases such as cancer.

### 2. Related Art

Signal transducer and activator of transcription 3 (STAT3) is one of a 7-member transcription factor family (STAT1, 2, 3, 4, 5A, 5B, and 6) that is activated in response to extracellular signals including cytokines, growth factors, and hormones. It modulates a large repertoire of genes involved in a number of critical functions: inflammation, cellular proliferation, survival and apoptosis, angiogenesis, transformation, as well as tumor invasion and metastasis (Bromberg et al., 1999). Constitutively active STAT3 is found in 50-100% of many different types of cancers and has been associated with poor prognosis (Bromberg et al., 1999; Levy and Darnell, 2002; Hsieh et al., 2005; Lieblein et al., 2008; and Marotta et al., 2011). Multiple studies suggest that targeting STAT3 in cancer suppresses cell growth and induces apoptosis in vitro and in vivo (Darnell, 2005; Jing and Tweardy, 2005; Leeman et al., 2006; Germain and Frank, 2007; Zhang et al., 2007; Egloff and Grandis, 2009; and Leeman-Neill et al., 2010) making it an attractive therapeutic target for cancer treatment (Debnath et al., 2012; and Darnell, 1997).

In the 1BG1 crystal structure of the core fragment of activated STAT3 homodimer (residues 138-722), each monomer subunit contains four distinct structural domains: an N-terminal four-helix bundle (residues 138-320), an eight-stranded β-barrel (residues 321-465), an α-helical “linker” domain (residues 466-585), an SH2 domain (residues 586-690), and a loop domain (residues 691-715) (Becker et al., 1998). The loops within the β-barrel and linker domains are responsible for DNA sequence specificity. The loop domain is phosphorylated on Tyr-705 and the pY₇₀₅-peptide motif within each monomer binds in trans to the SH2 domain of the other monomer, leading to dimerization. The N-terminal domain (residues 1-130; not shown in the crystal structure) is involved in oligomerization, which facilitates binding of multiple STAT3 dimers to two or more adjacent STAT3 DNA-binding elements within a gene promoter. The C-terminal domain—residues 716-722 in STAT3β and residues 716-770 in STAT3a (not shown in the crystal structure)—are involved in nuclear retention and transcriptional activation, respectively (Wen et al., 1995; Vinkemeier et al., 1996; and Xu et al., 1996). The SH2 domain is critical for STAT3's transcriptional function due to its requirement for recruitment to ligand-activated receptor complexes and dimerization (Bromberg and Darnell, 2000; Ren et al., 2003; and Darnell, 1997). Due to the moderately high affinity and specificity of SH2 binding to its cognate pY-peptide ligand motifs, targeting the SH2 domain is among the most viable strategies for inhibit STAT3 signaling.

The first STAT3 inhibitors identified were phosphotyrosylated (pY) peptides derived from peptide sequences shown to be bound by the STAT3 SH2 domain, such as P-pY₇₀₅LKTK within the C-terminal of STAT3 (Turkson et al., 2001) and pY₉₀₅LPQTV within gp130 (Ren et al., 2003). However, proteolytic cleavage resulting in short plasma half-life, along with poor oral bioavailability and low cell-membrane permeability have limited the clinical development of peptide inhibitors (Diao and Meibohm, 2013). To achieve better pharmacokinetic properties, peptidomimetic inhibitors derived from STAT3 SH2 pY-peptide ligands have been developed (Siddiquee et al., 2007; Turkson et al., 2004; and Chen et al., 2010). Among them, the conformationally constrained peptidomimetic, CJ-887 and its derivatives, based on pY₉₀₅LPQTV, which achieved high binding affinity as reflected in K, values as low as 15 nM (Chen et al., 2010). Similar to pY-peptides, however, lack of cell permeability and poor drug-like properties remain major obstacles for these compounds to be further developed for clinical use. Small molecules with favorable drug-like properties and high potency are highly sought after and great effort has been made to identify such inhibitors for STAT3 (Debnath et al., 2012). Structure-based virtual ligand screenings (SB-VLS) has been performed, and a number of active hits identified and lead compounds developed from them (Siddiquee et al., 2007; Song et al., 2005; and Xu et al., 2009). However, many exhibit weak binding affinities for STAT3 and evidence of their clinical effect has yet to be obtained for the few lead compounds that have entered into clinical trials (Bharadwaj et al., 2016).

One possible reason for the inefficiency of SB-VLS to identify hit compounds that bind STAT3 with high-affinity may be the high mobility of the STAT3 SH2 domain. In the crystal structure of dimers of the core STAT3 protein bound to DNA, the phosphopeptide binding region within the SH2 domain is resolved only to only ˜20 Å due to conformational flexibility (Becker et al., 1998). In addition, the crystal structure provides only a static snapshot of the domain's structure, which may be close to the “real” conformation in solution for a rigid domain, but may differ substantially from the structure in solution of a highly flexible domain, such as the SH2 domain of STAT3. Of note, the conformational flexibility of a protein is closely related to its functional activity and confirmation changes occur commonly in many types of protein (Shen et al., 2016; and Shen et al., 2017). Furthermore, in some cases, the induced binding pocket exhibits a more druggable site than its rigid counterpart and is of higher yield in drug design and discovery (Jiang et al., 2017; and Hocker et al., 2013).

Towards this end, molecular dynamics (MD) simulations of the STAT3 SH2 domain were conducted in a complex with CJ-887 and it was found that it induced protein conformation changes that favor ligand binding. An averaged structure from MD trajectory was calculated and used as a receptor model for SB-VLS that takes protein flexibility into consideration. Based on this “induced-active site” strategy, in silico screening followed by re-docking, re-scoring, selection for hit compounds that directly interact with pY+0 binding pocket, and testing for STAT3 targeting in vitro and in vivo, six compounds were identified as low micromolar inhibitors of cytokine-induced STAT3 in cells (2.7-34.5 μM). Two of these compounds are of high potency, low molecular weight, and fulfill Lipinski's rule of five, and, thus, would serve as excellent starting points for lead optimization.

### SUMMARY

In some aspects, the present disclosure provides pharmaceutical compositions comprising:

(A) a compound of the formula:

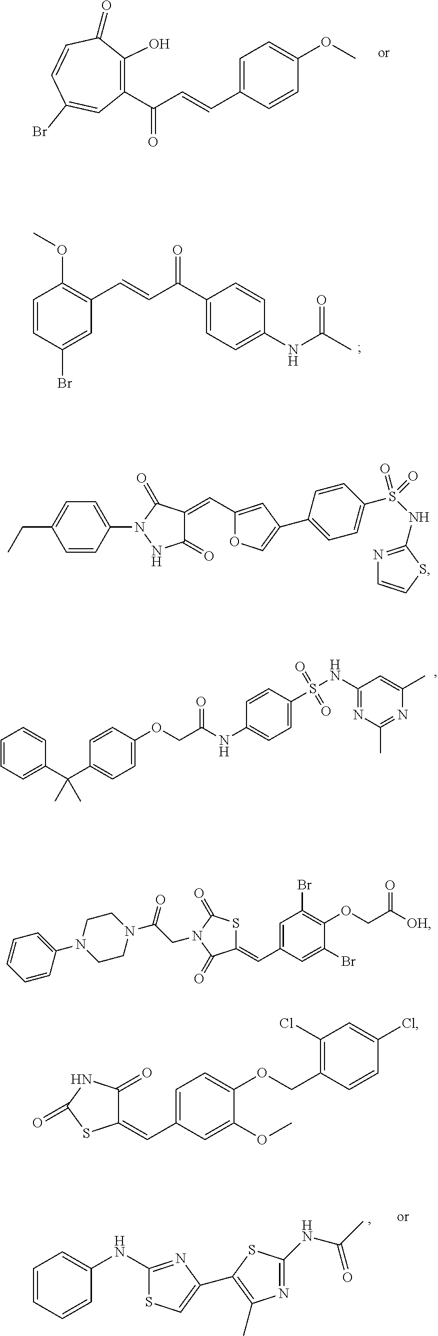

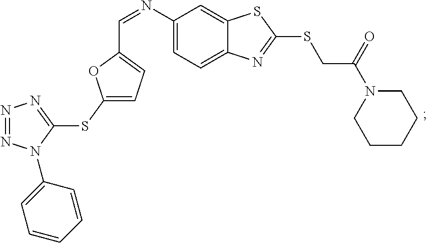 [SMILES: O=C(CSc1nc2ccc(/N=C\c3ccc(Sc4nnnn4-c4ccccc4)o3)cc2s1)N1CCCCC1]

or a pharmaceutically acceptable salt thereof; and

(B) an excipient.

In some embodiments, the compound is present in a therapeutically effective amount. In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition is formulated for oral administration. In other embodiments, the pharmaceutical composition is formulated for administration via injection. In further embodiments, the pharmaceutical composition is formulated for intraarterial administration, intramuscular administration, intraperitoneal administration, or intravenous administration. In some embodiments, the pharmaceutical composition is formulated as a unit dose.

In another aspect, the present disclosure provides methods of inhibiting STAT3 in a cell comprising contacting the cell with an effective amount of a compound of the formula:

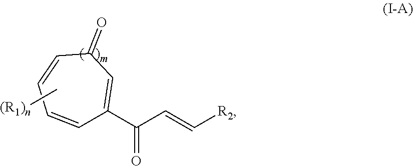

wherein:
n is 0, 1, 2, or 3;
m is 0 or 1;
R₁ is, in each instance independently, hydrogen, halo, hydroxy, amino, cyano, or nitro; or
   alkyl_{(C≤6)}, alkylamino_{(C≤6)}, dialkylamino_{(C≤6)}, alkoxy_{(C≤6)}, acyloxy_{(C≤6)}, amido_{(C≤6)}, or a substituted version of any of these groups; and
R2 is aryl_{(C≤8)}, substituted aryl_{(C≤8)}, heteroaryl_{(C≤8)}, or substituted heteroaryl_{(C≤8)};
   provided the compound is not:

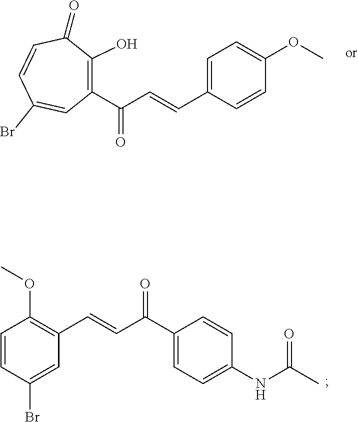 [SMILES: COc1ccc(/C=C/C(=O)c2cc(Br)ccc(=O)c2O)cc1 ; COc1ccc(Br)cc1/C=C/C(=O)c1ccc(NC(C)=O)cc1]

or
a compound of the formula:

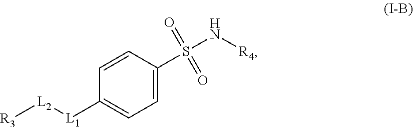

wherein:
L₁ is arenediyl_{(C≤8)}, heteroarenediyl_{(C≤8)}, NHC(O)alkanediyl_{(C≤6)}-O—, or a substituted version of any of these groups;
L₂ is arenediyl_{(C≤8)} or substituted arenediyl_{(C≤8)}; or a group of the formula:

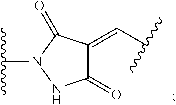 [SMILES: */C=C1\C(=O)NN(*)C1=O]

R₃ is aryl_{(C≤8)}, aralkyl_{(C≤8)}, heteroaryl_{(C≤8)}, heteroaralkyl_{(C≤8)}, or a substituted version of any of these groups; and
R₄ is aryl_{(C≤8)}, substituted aryl_{(C≤8)}, heteroaryl_{(C≤8)}, or substituted heteroaryl_{(C≤8)};
   provided the compound is not:

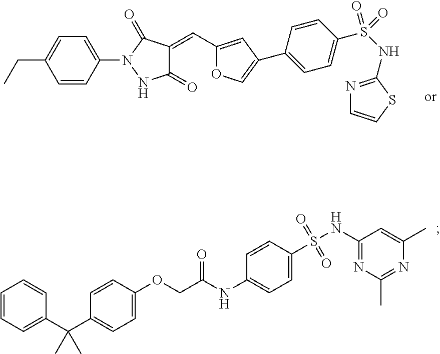 [SMILES: CCc1ccc(N2NC(=O)/C(=C\c3cc(-c4ccc(S(=O)(=O)Nc5nccs5)cc4)co3)C2=O)cc1 ; Cc1cc(NS(=O)(=O)c2ccc(NC(=O)COc3ccc(C(C)(C)c4ccccc4)cc3)cc2)nc(C)n1]

or
a compound of the formula:

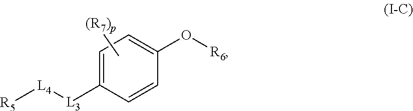

wherein:
p is 0, 1, or 2;
L₃ is a group of the formula:

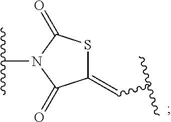

L₄ is a covalent bond, heterocycloalkanediyl_{(C≤8)}-C(O)-alkanediyl_{(C≤6)}-, or substituted -heterocycloalkanediyl_{(C≤8)}-C(O)-alkanediyl_{(C≤6)}-;
R₅ is hydrogen, aryl_{(C≤8)}, substituted aryl_{(C≤8)}, heteroaryl_{(C≤8)}, or substituted heteroaryl_{(C≤8)};
R₆ is alkyl_{(C≤6)}, substituted alkyl_{(C≤8)}, aralkyl_{(C≤6)}, or substituted aralkyl_{(C≤8)}; and
R₇ is, in each instance independently, hydrogen, halo, hydroxy, amino, cyano, or nitro; or alkyl_{(C≤6)}, alkylamino_{(C≤6)}, dialkylamino_{(C≤6)}, alkoxy_{(C≤6)}, acyloxy_{(C≤6)}, amido_{(C≤6)}, or a substituted version of any of these groups;
   provided that the compound is not:

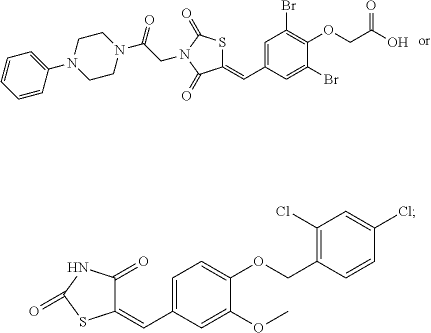 [SMILES: O=C(O)COc1c(Br)cc(/C=C2\SC(=O)N(CC(=O)N3CCN(c4ccccc4)CC3)C2=O)cc1Br ; COc1cc(/C=C2/SC(=O)NC2=O)ccc1OCc1ccc(Cl)cc1Cl]

or
a compound of the formula:

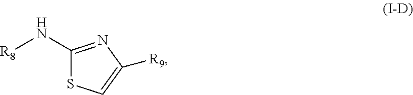

wherein:
R₈ and R₉ are each independently aryl_{(C≤8)}, substituted aryl_{(C≤8)}, heteroaryl_{(C≤8)}, or substituted heteroaryl_{(C≤8)};
   provided the compound is not:

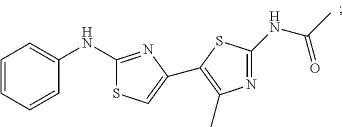 [SMILES: CC(=O)Nc1nc(C)c(-c2csc(Nc3ccccc3)n2)s1]

or
a compound of the formula:

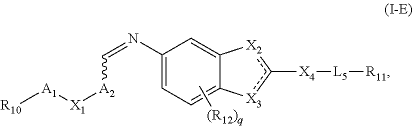

wherein:
q is 0, 1, 2, or 3;
A₁ and A₂ are each independently arenediyl_{(C≤8)}, substituted arenediyl_{(C≤8)}, heteroarenediyl_{(C≤8)}, or substituted heteroarenediyl_{(C≤8)};
L₅ is a covalent bond, —C(O)—, -alkanediyl_{(C≤6)}-C(O)—, or substituted -alkanediyl_{(C≤6)}-C(O)—;
R₁₀ is alkyl_{(C≤6)}, aryl_{(C≤8)}, aralkyl_{(C≤8)}, or substituted version of any of these groups;
R₁₁ is alkyl_{(C≤6)}, cycloalkyl_{(C≤8)}, heterocycloalkyl_{(C≤8)}, or substituted version ofany of these groups;
R¹² is, in each instance independently, hydrogen, halo, hydroxy, amino, cyano, or nitro; or
X₁ and X₄ are each independently —O—, —S—, or —NRₐ—, wherein:
   Rₐ is hydrogen, alkyl_{(C≤6)}, or substituted alkyl_{(C≤6)}; and
X₂ and X₃ are each independently —O—, —S—, —N═, —NR_{b}—, wherein:
   R_{b} is hydrogen, alkyl_{(C≤6)}, or substituted alkyl_{(C≤6)};
      provided that one of X₂ or X₃ is not —N═ and provided the compound is not:

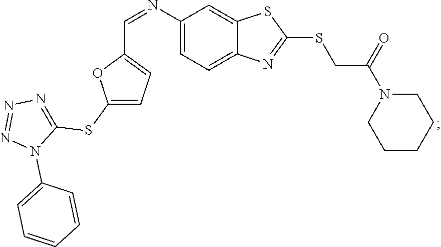 [SMILES: O=C(CSc1nc2ccc(/N=C\c3ccc(Sc4nnnn4-c4ccccc4)o3)cc2s1)N1CCCCC1]

or a pharmaceutically acceptable salt of these formulae.

In some embodiments, the compound is of formula I-A. In other embodiments, the compound is of formula I-B. In still other embodiments, the compound is of formula I-C. In yet other embodiments, the compound is of formula I-D. In other embodiments, the compound is of formula I-E. In some embodiments, m is 0. In other embodiments, m is 1. In some embodiments, n is 0, 1, or 2. In further embodiments, 1 or 2. In some embodiments, n is 1. In other embodiments, n is 2. In some embodiments, R₁ is hydroxy. In other embodiments, R₁ is halo, such as bromo. In still other embodiments, R₁ is amido_{(C≤6)} or substituted amido_{(C≤6)}. In further embodiments, R₁ is amido_{(C≤6)}, such as acetamido. In some embodiments, R₂ is aryl_{(C≤8)} or substituted aryl_{(C≤8)}. In further embodiments, R₂ is substituted aryl_{(C≤8)}, such as 4-methoxyphenyl or 5-bromo-2-methoxyphenyl. In some embodiments, L₁ is heteroarenediyl_{(C≤8)} or substituted heteroarenediyl_{(C≤8)}. In further embodiments, L₁ is heteroarenediyl_{(C≤8)}, such as furan-3,5-diyl. In other embodiments, L₁ is —NHC(O)-alkanediyl_{(C≤6)}-O— or substituted —NHC(O)-alkanediyl_{(C≤6)}-O—. In further embodiments, L₁ is —NHC(O)-alkanediyl_{(C≤6)}-O—, such as —NHC(O)CH₂O—. In some embodiments, L₂ is aryl_{(C≤8)} or substituted aryl_{(C≤8)}. In further embodiments, L₂ is aryl_{(C≤8)}, such as benzen-1,4-diyl. In other embodiments, L₂ is a group of the formula:

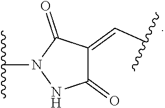 [SMILES: */C=C1\C(=O)NN(*)C1=O]

In some embodiments, R₃ is aryl_{(C≤8)} or substituted aryl_{(C≤8)}. In further embodiments, R₃ is aryl_{(C≤8)}, such as R₃ is 4-ethylphen-1-yl. In other embodiments, R₃ is aralkyl_{(C≤8)} or substituted aralkyl_{(C≤8)}. In further embodiments, R₃ is aralkyl_{(C≤8)}, such as 1,1-dimethyl-1-phenylmethyl. In some embodiments, R₄ is heteroaryl_{(C≤8)} or substituted heteroaryl_{(C≤8)}. In further embodiments, R₄ is heteroaryl_{(C≤8)}, such as thiazol-2-yl or 2,6-dimethylpyrimidin-4-yl. In some embodiments, L₄ is a covalent bond. In other embodiments, L₄ is -heterocycloalkanediyl_{(C≤8)}-C(O)-alkanediyl_{(C≤6)}- or substituted -heterocycloalkanediyl_{(C≤8)}-C(O)-alkanediyl_{(C≤6)}-. In further embodiments, L₄ is -heterocycloalkanediyl_{(C≤8)}-C(O)-alkanediyl_{(C≤6)}-, such as -piperazin-1,4-diyl-C(O)CH₂—. In some embodiments, R₅ is hydrogen. In other embodiments, R₅ is aryl_{(C≤8)} or substituted aryl_{(C≤8)}. In further embodiments, R₅ is aryl_{(C≤8)}, such as phenyl. In some embodiments, R₆ is alkyl_{(C≤6)} or substituted alkyl_{(C≤8)}. In further embodiments, R₆ is substituted alkyl_{(C≤8)}, such as carboxymethyl. In other embodiments, R₆ is aralkyl_{(C≤6)} or substituted aralkyl_{(C≤8)}. In further embodiments, R₆ is substituted aralkyl_{(C≤8)}, such as R₆ is 2,4-dichlorophenyl. In some embodiments, p is 1 or 2. In some embodiments, p is 1. In other embodiments, p is 2. In some embodiments, R₇ is halo, such as bromo. In other embodiments, R₇ is alkoxy_{(C≤6)} or substituted alkoxy_{(C≤6)}. In further embodiments, R₇ is alkoxy_{(C≤6)}, such as methoxy.

In some embodiments, R₈ is aryl_{(C≤8)} or substituted aryl_{(C≤8)}. In further embodiments, R₈ is aryl_{(C≤8)}, such as phenyl. In some embodiments, R₉ is heteroaryl_{(C≤8)} or substituted heteroaryl_{(C≤8)}. In further embodiments, R₉ is substituted heteroaryl_{(C≤8)}, such as 2-acetamido-4-methylthiazol-5-yl. In some embodiments, X₁ is —S—. In some embodiments, X₄ is —S—. In some embodiments, X₂ is —S—. In some embodiments, X₃ is —N═. In some embodiments, A₁ is heteroarenediyl_{(C≤8)} or substituted heteroarenediyl_{(C≤8)}. In further embodiments, A1 is heteroarenediyl_{(C≤8)}, such as tetrazol-1,5-diyl. In some embodiments, A₂ is heteroarenediyl_{(C≤8)} or substituted heteroarenediyl_{(C≤8)}. In further embodiments, A₂ is heteroarenediyl_{(C≤8)}, such as furan-2,5-diyl. In some embodiments, L₅ is -alkanediyl_{(C≤6)}-C(O)— or substituted -alkanediyl_{(C≤6)}-C(O)—. In further embodiments, L₅ is -alkanediyl_{(C≤6)}-C(O)—, such as —CH₂C(O)—. In some embodiments, R₁₀ is aryl_{(C≤8)} or substituted aryl_{(C≤8)}. In further embodiments, R₁₀ is aryl_{(C≤8)}, such as phenyl. In some embodiments, Ru is heterocycloalkyl_{(C≤8)} or substituted heterocycloalkyl_{(C≤8)}. In further embodiments, Ru is heterocycloalkyl_{(C≤8)}, such as N-piperidinyl. In some embodiments, q is 0.

In still another aspect, the present disclosure provides methods of inhibiting STAT3 in a cell comprising contacting the cell with an effective amount of a pharmaceutical composition of the present disclosure or a compound of the formula:

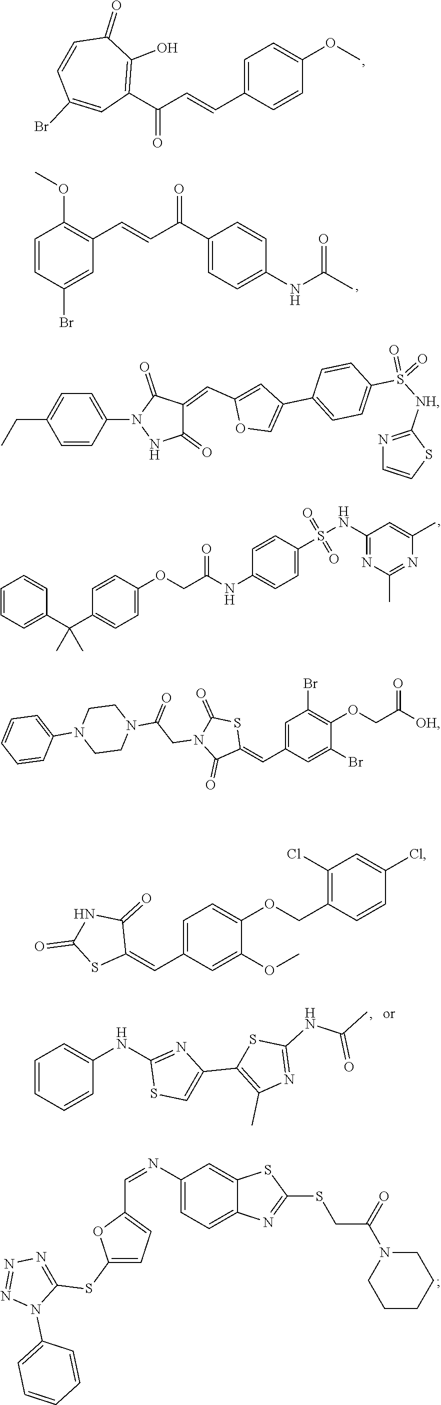

or a pharmaceutically acceptable salt thereof.

In some embodiments, the cell is an immune cell or a cancer cell. In some embodiments, the cell is a cancer stem cell. In some embodiments, the method is further defined as a method of treating a subject and comprising administering an effective amount of a pharmaceutical formulation comprising the compound to the subject. In some embodiments, the subject has an autoimmune disease, an inflammatory disease of a cancer. In some embodiments, the inflammatory disease is atherosclerosis, peripheral vascular disease, coronary artery disease, hypertension, osteoporosis, type 2 diabetes or dementia. In some embodiments, the subject has a cancer. In some embodiments, the cancer is a metastatic cancer. In some embodiments, the cancer overexpresses STAT3 or exhibits increased STAT3 activation. In some embodiments, the cancer is a breast cancer. In some embodiments, the cancer is a carcinoma or a hematological cancer. In some embodiments, the cancer is leukemia. In some embodiments, the cancer is acute myeloid leukemia (AML). In some embodiments, the method further comprises administering a further anti-cancer therapy to the subject. In some embodiments, the further anti-cancer therapy is chemotherapy, radiotherapy, gene therapy, surgery, hormonal therapy, anti-angiogenic therapy or cytokine therapy. In some embodiments, the further anti-cancer therapy is an immune check point inhibitor therapy.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows an overview of the structure-based virtual screening strategy that incorporates SH2 domain flexibility. CJ-887 was docked to a monomer of STAT3 from the crystal structure 1BG1, followed by 10 ns molecular dynamics simulation to relax the complex structure. The averaged structure from MD simulation was used as receptor, to perform two-layer docking experiments by using Glide HTVS and SP parameter sets, respectively. Structural criteria, including binding at pY+0 pocket, were used to filter the docking poses for compound selection. The shortlisted compounds were screened for anti-STAT3 properties as well as ability to inhibit growth of pSTAT3-dependent breast cancer cell lines.

FIGS. 2A-2C show binding pose of phosphorylated peptide fragment with STAT3 SH2 domain from the crystal structure with PDB code 1BG1 (Becker et al., 1998) (FIGS. 2A & 2B) and the docking pose of CJ-887 (FIG. 2C). The protein is shown in solvate accessible surface model in a cartoon model in B and C for clarity. Residues in 5 Å of ligands are shown in line whereas the ligands are drawn in stick model. Non-polar hydrogen atoms are hidden. Carbon atoms are colored in green and orange in protein and ligands, separately. Oxygen atoms are colored in red and nitrogen atoms in blue. Hydrogen bonds are shown in yellow dashed lines.

FIGS. 3A-3C show the averaged structure of STAT3 bound to CJ-887 (FIG. 3A) 2D structure of CJ-887 (FIG. 3B) CJ-887 in complex with STAT3 (466 to 716) from MD simulation and (FIG. 3C) the comparison of the docking pose and averaged structure. Residues within 5 Å of CJ-887 are drawn in lines while CJ-887 is rendered in stick model. The carbon atoms of protein in the docking pose and the averaged structure are colored in green and cyan, respectively. And the carbon atoms of CJ-887 in docking pose and averaged structure are colored in green and orange for clarity. Oxygen atoms are colored in red and nitrogen atoms in blue. Hydrogen bonds are shown in yellow dashed lines.

FIGS. 4A-4H show binding modes of the eight hits. Binding poses of compounds SPEC85/57/8/98/101/29/93/106 (FIGS. 4A-4H) in the averaged structure of STAT3 from MD simulation are illustrated. The left side of each panel shows the electrostatic molecular surface model of STAT3 SH2 domain with blue for positive charged and red for negative charged area; electrostatic potential calculated by APBS in Pymol (Baker et al., 2001). The right side of each panel is a clear view with residues in lines. Carbon atoms in the protein and ligands are colored in cyan and orange. Nitrogen and oxygen atoms are colored in blue and red. Non-polar hydrogen atoms are hidden for clarity. Predicted hydrogen bonds are shown in yellow dashed lines.

FIG. 5 shows inhibition of G-CSF stimulated pSTAT3 in Kasumi cells by SPEC compounds. Serum-starved (1 hour) Kasumi-1 cells, pre-incubated with compound/DMSO (0/0.1/0.3/1/3/10/100 μM, 1 hour), were treated with G-CSF (10 ng/ml, 15′). Total protein was assayed for pSTAT3 and GAPDH levels by Luminex. GAPDH-normalized pSTAT3 values were divided by the same for untreated cells and expressed as percentage. These values were plotted as a function of Log [M] compound, and IC₅₀ calculated using GraphPad. Data from representative experiments from at least two repeats is shown.

FIG. 6 shows inhibition of growth of pSTAT3-high breast cancer MDA-MB-468 cells by SPEC compounds. MDA-MB-468 cells were cultured for 48 hrs in complete DMEM with 10% FBS±compound (0/0.1/0.3/1/3/10/100 μM) in cell-culture-treated plates and viable cells quantitated using MTT. Relative % viability (viability after any treatment÷viability of untreated cells×100) was plotted as a function of Log [M] compound, and IC₅₀ values calculated using GraphPad. Data show representative experiments from ≥2 replicates. Mean IC₅₀ values are shown in Table 1.

FIG. 7 shows inhibition of growth of pSTAT3-high breast cancer MDA-MB-468 cells by SPEC compounds. MDA-MB-468 cells were cultured for 72 hrs in complete DMEM with 10% FBS±compound (0/0.1/0.3/1/3/10/100 μM) in cell-culture-treated ultra-low attachment 96-well plates and viable cells quantitated using MTT. Relative % viability (viability after any treatment viability of untreated cells×100) was plotted as a function of Log [M] compound, and IC₅₀ values calculated using GraphPad. Data show representative experiments from ≥2 replicates. Mean IC₅₀ values are shown in Table 1.

FIG. 8 shows inhibition of growth of pSTAT3-high breast cancer MDA-MB-231 cells by SPEC compounds. MDA-MB-231 cells were cultured for 48 hrs in complete DMEM with 10% FBS±compound (0/0.1/0.3/1/3/10/100 μM) in cell-culture-treated plates and viable cells quantitated using MTT. Relative % viability (viability after any treatment÷viability of untreated cells×100) was plotted as a function of Log [M] compound, and IC₅₀ values calculated using GraphPad. Data show representative experiments from ≥2 replicates. Mean IC₅₀ values are shown in Table 1.

FIG. 9 shows inhibition of growth of pSTAT3-high breast cancer MDA-MB-231 cells by SPEC compounds. MDA-MB-231 cells were cultured for 72 hrs in complete DMEM with 10% FBS±compound (0/0.1/0.3/1/3/10/100 μM) in cell-culture-treated ultra-low attachment 96-well plates and viable cells quantitated using MTT. Relative % viability (viability after any treatment÷viability of untreated cells×100) was plotted as a function of Log [M] compound, and IC₅₀ values calculated using GraphPad. Data show representative experiments from ≥2 replicates. Mean IC₅₀ values are shown in Table 1.

FIGS. 10A-10D show abilities of SPEC compounds to inhibit growth of pSTAT3-high breast cancer cell lines, correlate to their abilities to inhibit pSTAT3. The IC50s for the abilities of the eight compounds SPEC29/8/93/98/106/57/101/85 to inhibit anchorage dependent (FIGS. 10A & 10C) and anchorage independent (FIGS. 10B & 10D) growth of MDA-MB468 and MDA-MB-231 and the IC50s for inhibiting G-CSF-stimulated pSTAT3 were tested for correlation using non-parametric spearman correlation coefficient. Linear regression lines are shown along with Spearman (rank) correlation co-efficients and p values calculated using GraphPad Prism.

FIGS. 11A-11C show the comparison of the docking pose and averaged structure (FIG. 11A) overlap of docking pose (green) and average structure (cyan); (FIG. 11B) Docking pose of CJ-887 in crystal structure with protein shown in electrostatic surface model; (FIG. 11C) CJ-887 and SH2 complex in average structure with protein shown in electrostatic surface model. From blue to red, the charges change from negative to positive. With the movements of αA helix and K591 side chain, the shape of p Y+O pocket becomes larger to accommodate structural diverse compounds.

FIG. 12 shows inhibition of G-CSF stimulated pSTAT1 in Kasumi cells by SPEC compounds. Serum-starved (1 hour) Kasumi-1 cells, pre-incubated with compound/DMSO (0/0.1/0.3/1/3/10/100 μM, 1 hour), were treated with G-CSF (10 ng/ml, 15′). Total protein was assayed for pSTAT3 and GAPDH levels by Luminex. GAPDH-normalized pSTAT1 values were divided by the same for untreated cells and expressed as percentage. These values were plotted as a function of Log [M] compound, and IC₅₀ calculated using GraphPad. Data from representative experiments from at least two repeats is shown.

FIG. 13 shows inhibition of G-CSF stimulated pSTAT5 in Kasumi cells by SPEC compounds. Serum-starved (1 hour) Kasumi-1 cells, pre-incubated with compound/DMSO (0/0.1/0.3/1/3/10/100 μM, 1 hour), were treated with G-CSF (10 ng/ml, 15′). Total protein was assayed for pSTAT5 and GAPDH levels by Luminex. GAPDH-normalized pSTAT5 values were divided by the same for untreated cells and expressed as percentage. These values were plotted as a function of Log [M] compound, and IC₅₀ calculated using GraphPad. Data from representative experiments from at least two repeats is shown.

### DESCRIPTION OF ILLUSTRATIVE EMBODIMENTS

The present disclosure provides small-molecule STAT3 inhibitors. Also, provided herein are methods of using these compounds, such as for the treatment of cancer.

### I. Compounds and Synthetic Methods

TABLE A
Structure and compound ID numbers for small-molecule STAT3 inhibitors.

| SPEC-# | Structure |
|---|---|
| 8 | 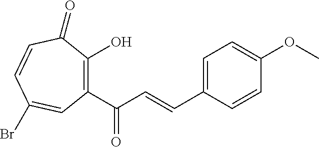 [SMILES: COc1ccc(/C=C/C(=O)c2cc(Br)ccc(=O)c2O)cc1] |
| 29 | 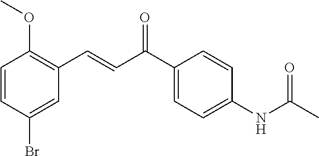 [SMILES: COc1ccc(Br)cc1/C=C/C(=O)c1ccc(NC(C)=O)cc1] |
| 57 | 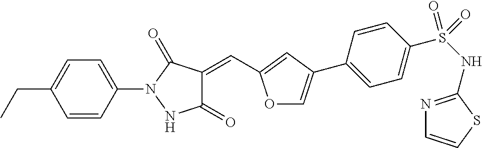 [SMILES: CCc1ccc(N2NC(=O)/C(=C\c3cc(-c4ccc(S(=O)(=O)Nc5nccs5)cc4)co3)C2=O)cc1] |
| 85 | 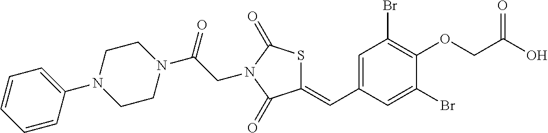 [SMILES: O=C(O)COc1c(Br)cc(/C=C2\SC(=O)N(CC(=O)N3CCN(c4ccccc4)CC3)C2=O)cc1Br] |
| 93 | 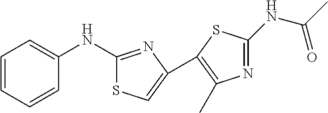 [SMILES: CC(=O)Nc1nc(C)c(-c2csc(Nc3ccccc3)n2)s1] |
| 98 | 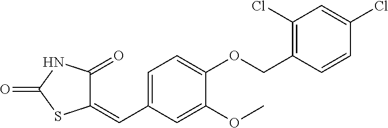 [SMILES: COc1cc(/C=C2/SC(=O)NC2=O)ccc1OCc1ccc(Cl)cc1Cl] |
| 101 | 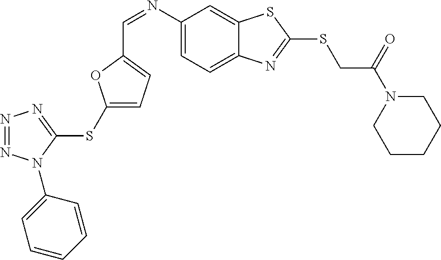 [SMILES: O=C(CSc1nc2ccc(/N=C\c3ccc(Sc4nnnn4-c4ccccc4)o3)cc2s1)N1CCCCC1] |
| 106 | 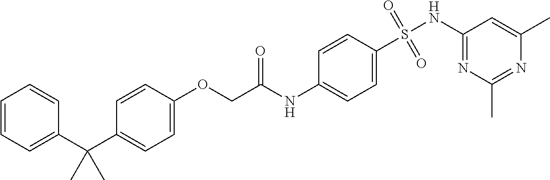 [SMILES: Cc1cc(NS(=O)(=O)c2ccc(NC(=O)COc3ccc(C(C)(C)c4ccccc4)cc3)cc2)nc(C)n1] |

The compounds of the present invention (also referred to as “compounds of the present disclosure”) are shown, for example, above, in Table A or in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### II. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### III. Treatment of Cancer and Other Hyperproliferative Diseases

While hyperproliferative diseases can be associated with any disease which causes a cell to begin to reproduce uncontrollably, the prototypical example is cancer. Psoriasis is another example. One of the key elements of cancer is that the cell's normal apoptotic cycle is interrupted and thus agents that interrupt the growth of the cells are important as therapeutic agents for treating these diseases. In some embodiments, the STAT3 inhibitors described herein may be used to decreased cell counts and as such may be used to treat a variety of cancers or other malignancies.

In some embodiments, cancer, cancer tissue, or cancer cells may be treated by the compounds, methods, and compositions disclosed herein. In some embodimantes, cancer cells or tissue that may be treated include but are not limited to cells or tissue from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In some embodiments, the cancer that may be treated may be of the following histological types: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia, including hairy cell leukemia; chronic lymphocytic leukemia (CLL); acute lymphoblastic leukemia (ALL); acute myeloid leukemia (AML); and chronic myeloblastic leukemia.

In another aspect, the compounds, compositions, and methods disclosed herein may be used to treat cancer or other hyperproliferative diseases. While hyperproliferative diseases can be associated with any disease which causes a cell to begin to reproduce uncontrollably, the prototypical example is cancer. One of the elements of cancer is that the cell's normal apoptotic cycle is interrupted. As such, agents that interrupt the growth of the cells are important as therapeutic agents for treating these diseases. In this disclosure, the compounds of the present disclosure thereof may be used to lead to decreased cell counts and may be used to treat a variety of types of cancer.

In some embodiments, cancer cells that may be treated with the compounds or compositions of the present disclosure include, but are not limited to, bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, and uterus cells.

In some embodiments, tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

In certain embodiments regarding methods of treating cancer in a patient, comprising administering to the patient a pharmaceutically effective amount of a compound of the present disclosure, the pharmaceutically effective amount is 0.1-1000 mg/kg. In certain embodiments, the pharmaceutically effective amount is administered in a single dose per day. In certain embodiments, the pharmaceutically effective amount is administered in two or more doses per day. The compound may be administered by contacting a tumor cell during ex vivo purging, for example. The method of treatment may comprise any one or more of the following: a) inducing cytotoxicity in a tumor cell; b) killing a tumor cell; c) inducing apoptosis in a tumor cell; d) inducing differentiation in a tumor cell; or e) inhibiting growth in a tumor cell. The tumor cell may be any type of tumor cell, such as a brain cell. Other types of cells include, for example, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In some embodiments, treatment methods further comprise monitoring treatment progress. In some of these embodiments, the method includes the step of determining a level of changes in hematological parameters and/or cancer stem cell (CSC) analysis with cell surface proteins as diagnostic markers or diagnostic measurement (e.g., screen, assay) in a patient suffering from or susceptible to a disorder or symptoms thereof associated with cancer in which the patient has been administered a therapeutic amount of a compound or composition as described herein. The level of the marker determined in the method can be compared to known levels of marker in either healthy normal controls or in other afflicted patients to establish the patient's disease status. In some embodiments, a second level of the marker in the patient is determined at a time point later than the determination of the first level, and the two levels are compared to monitor the course of disease or the efficacy of the therapy. In some embodiments, a pre-treatment level of marker in the patient is determined prior to beginning treatment according to the methods described herein; this pre-treatment level of marker can then be compared to the level of marker in the patient after the treatment commences, to determine the efficacy of the treatment.

In some embodiments, the patient is a mammal, e.g., a primate, preferably a higher primate, e.g., a human (e.g., a patient having, or at risk of having, a disorder described herein). In some embodiments, the patient is in need of enhancing the patient's immune response. In certain embodiments, the patient is, or is at risk of being, immunocompromised. For example, in some embodiments, the patient is undergoing or has undergone a chemotherapeutic treatment and/or radiation therapy. Alternatively, or in combination, the patient is, or is at risk of being, immunocompromised as a result of an infection.

### IV. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

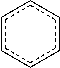

covers, for example,

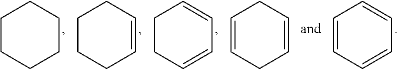

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

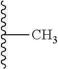

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

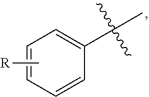

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

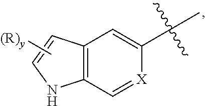

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn−n″” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic 11 system. An aromatic compound or chemical group may be depicted as a single resonance structure; however, depiction of one resonance structure is taken to also refer to any other resonance structure. For example:

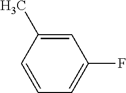

is also taken to refer to

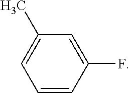

Aromatic compounds may also be depicted using a circle to represent the delocalized nature of the electrons in the fully conjugated cyclic 1 L system, two non-limiting examples of which are shown below:

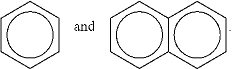

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

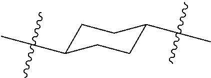 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

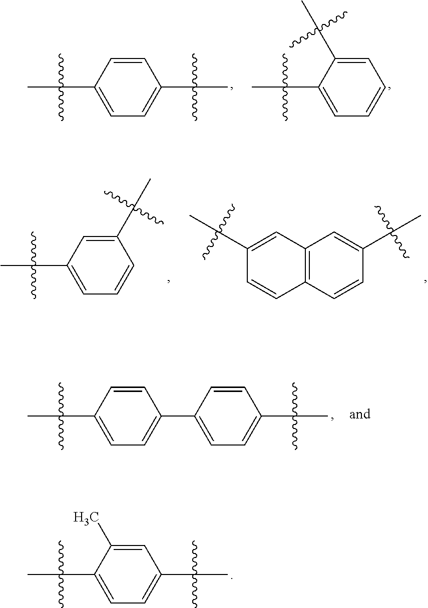

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

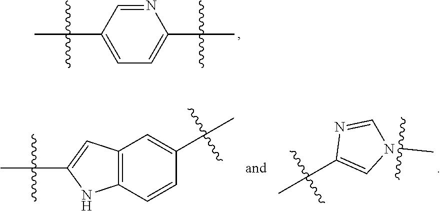

A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, aryl, aralkyl, heteroaryl, heterocycloalkyl, and acyl, respectively.

The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Inhibition of STAT3 with Compounds Identified in Library Screen

### A. Overview

Efforts to develop STAT3 inhibitors have focused on its SH2 domain starting with short phosphotyrosylated peptides based on STAT3 binding motifs, e.g. pY₉₀₅LPQTV within gp130. Despite binding to STAT3 with high affinity, issues regarding stability, bioavailability, and membrane permeability of these peptides, as well as peptidomimetics such as CJ-887, have limited their further clinical development and led to increased interest in small-molecule inhibitors. Some small molecule STAT3 inhibitors have been identified using structure-based virtual ligand screening (SB-VLS); while having favorable drug-like properties, most suffer from weak binding affinities, possibly due to the high flexibility of the target domain, especially within the region involved in pY-peptide binding. Described herein are molecular dynamic (MD) simulations of the SH2 domain in a complex with CJ-887, with a focus on ligand-induced protein conformation changes that increase binding affinity. An averaged structure from this MD trajectory was used as “induced-active site” receptor model for SB-VLS of 110,000 compounds within the SPEC database. Screening was followed by re-docking and re-scoring of the top 30% of hits, selection for hit compounds that directly interact with pY+0 binding pocket residues R609-S614, and testing them for STAT3 targeting in vitro and in vivo, which identified two lead hits with good activity and favorable drug-like properties. Unlike most STAT3 inhibitors previously identified containing negatively-charged moieties that mediate binding the pY+0 binding pocket, these compounds are uncharged and likely will serve as good candidates for anti-STAT3 drug development.

### B. Material and Methods

Cell Lines: Breast cancer cell lines MDA-MB-231 and MDA-MB-468 and the AML line Kasumi-1 were obtained from the cell line core at BCM and ATCC respectively, and cultured in complete DMEM or RPMI respectively, with 10% FBS, antibiotics penicillin, streptomycin and amphotericin.

Molecular docking: The STAT3 SH2 domain was taken from x-ray crystal structure 1BG1 (Becker et al., 1998) stored in Protein Data Bank (PDB). Only residues 466 to 716 of the monomer, including the linker domain, SH2 domain and the loop bearing Tyr-705, were kept. Residues 1-465, including the N-terminal DNA-binding, 4-helix bundle and β-barrel domains, were removed since they do not have direct interactions with the SH2 domain and the dimerization interface. The missing residues 689-701 in the crystal structure were constructed and minimized by Prime with OPLS force field. The modeled structure was subjected to Protein Preparation Wizard workflow in Maestro 9.2. Bond orders were assigned and all hydrogen atoms were minimized to reach the convergence of RMSD=0.3 Å with OPLS force field. A grid-enclosing box was centered on the GLU 638 to enclose residues located within 20 Å, where the phosphorylated peptide located. A scaling factor of 1.0 was set to van der Waals (VDW) radii of those receptor atoms with partial atomic charge less than 0.25. The “Builder” module in Schrodinger was used to build the molecular structure of CJ-887 and OPLS force field was applied to obtain the minimized energy structure. LigPrep (LigPrep v. Schrodinger, L I C., 2011) module was performed to get the diverse conformation of CJ-887 with pH value of 7.0±2.0.

Molecular dynamics simulation: To relax the docking pose of CJ-887, molecular dynamics simulation was performed for the STAT3-CJ-887 complex. RED was used to do charge derivation in cooperation with Gaussian 09 program at the b3lyp/6-31G* level for the ligand (Cieplak et al., 1995; and Dupradeau et al., 2010). TLEaP was used to solvate the systems in a truncated-octahedron box of TIP3P water molecules (Jorgensen et al., 1983) with 10 Å of minimum distance from the atoms of protein or ligand to the border of the box. Na+ atoms were added to obtain an electrically neural system. MD simulations were carried out with AMBERI1 (Case et al., 2010) on BlueBiou of IBM supercomputing cluster in Rice University. The standard amber ff99 force field (Hornak et al., 2006) and the general AMBER force field (GAFF) (Wang et al., 2004) was used as the parameter for protein and ligand, respectively. Calculations employed the particle mesh Ewald method (Darden et al., 1993) to treat the long-range electrostatic interactions with periodic boundary conditions and a cut-off value of 10 Å. All bonds containing hydrogen atoms were constrained using the SHAKE algorithm (Ryckaert et al., 1977) along with time step of 2 fs. The temperature is controlled by Langevin thermostat (Izaguirre et al., 2001). Energy minimization was executed by the steepest descent method for the first 2,000 steps and followed by the conjugated gradient method for the second 2,000 steps with 5 kcal/mol*Å² restraints on all the atoms of protein and ligand. The temperature was increased from OK to 300K gradually over 40 ps with 10 kcal/mol*Å² restraints on the solvent, and then the force constant was decreased to 5, 1 and 0 kcal/mol*Å², respectively, for the following three 40 ps simulations, using the NTP ensemble to relax the water molecules and Na⁺ ions. 10 ns production simulation was done at 1 atm and 300 K under the NTP ensemble with a time step of 2 fs.

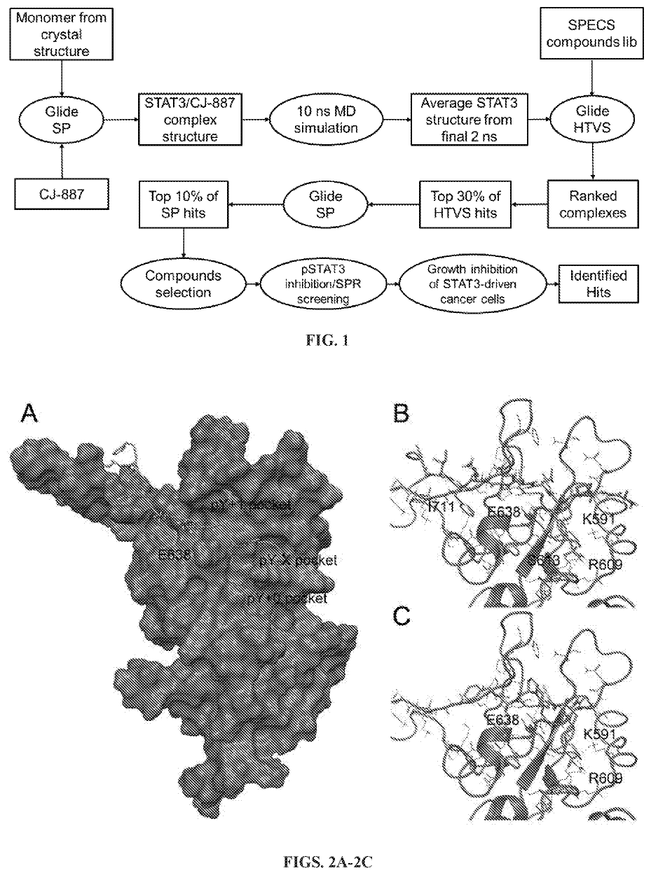

Virtual ligand screening protocol: All molecular structures from SPECS screening compounds library were prepared using LigPrep in Schrodinger to add hydrogen atoms and predict diverse protonation states within the scale of pH value from 5.4 to 9.4. Enumeration of Stereoisomers and tautomers was also taken into consideration during the ligand prepare process. The averaged structure obtained from MD simulation was prepared by using the Protein Preparation Workflow in Maestro (Maestro v. Schrodinger, L I C., 2011). Bond orders were assigned and all hydrogen atoms were minimized to reach the convergence of RMSD=0.3 Å with OPLS force field. The grid generation process was the same as the protocol described above for the CJ-887 docking. The high throughput virtual screening (HTVS) mode of Glide (Glide v. Schrodinger, L I C., 2011) was used to explore the binding modes and evaluate the binding affinities for all the compounds in the active site with default settings. We redocked and rescored thirty percent of top-ranking molecules with standard precision (SP) parameter settings. Top 10% poses from the SP docking experiment served as the inputs for pose filter. Criteria that were applied to do the molecule selection were (1) hydrogen bonding with at least one of the pY+0 pocket residues including R609, S611-5614 and (2) structural diversity in chemical space. The whole scheme of hit discovery, enumerating the docking and simulation strategies as well as the functional screens of potential in-silico hits, is stated in FIG. 1.

Surface plasmon resonance (SPR) assay of STAT3 binding pY-peptide. STAT3 (aa 127-722) at a concentration of 200 nM in 20 mM Tris buffer (pH 8) was pre-incubated without or with compounds prior to injection onto an SA chip immobilized with phosphorylated and control non-phosphorylated biotinylated EGFR derived dodecapeptides based on sequence surrounding Y1068 (Shao et al., 2004), on a Biacore 3000 biosensor (Biacore inc., Piscataway N.J.) and analyzed as described (Bharadwaj et al., 2016).

Luminex bead-based assay. Luminex bead-based assays were used to determine levels of pSTAT3, pSTAT1, pSTAT5, and GAPDH, as described (Bharadwaj et al., 2015).

Anchorage-independent and dependent cell growth. Cells were cultured in triplicates in complete DMEM±drug, in ultra-low attachment 96 well plates for 72 hrs or cell-culture treated plates for 48 hrs and viable cells were quantitated using MTT. Optical density (OD) was measured at 590 nm using a 96-well multi-scanner (Synergy H₁ microplate reader, BioTek Inc, VT, USA). Relative % viability (viability after any treatment÷viability of untreated cells×100) was plotted along Y-axis. At least 2 replicates experiments were performed and were used for IC50 calculation using GraphPad software.

### C. Results

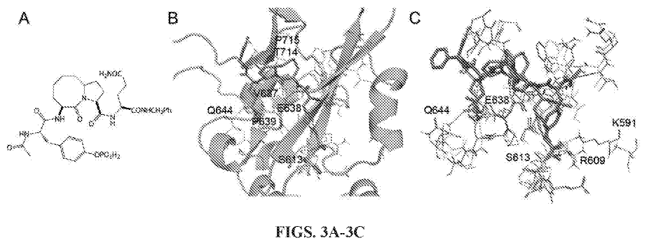

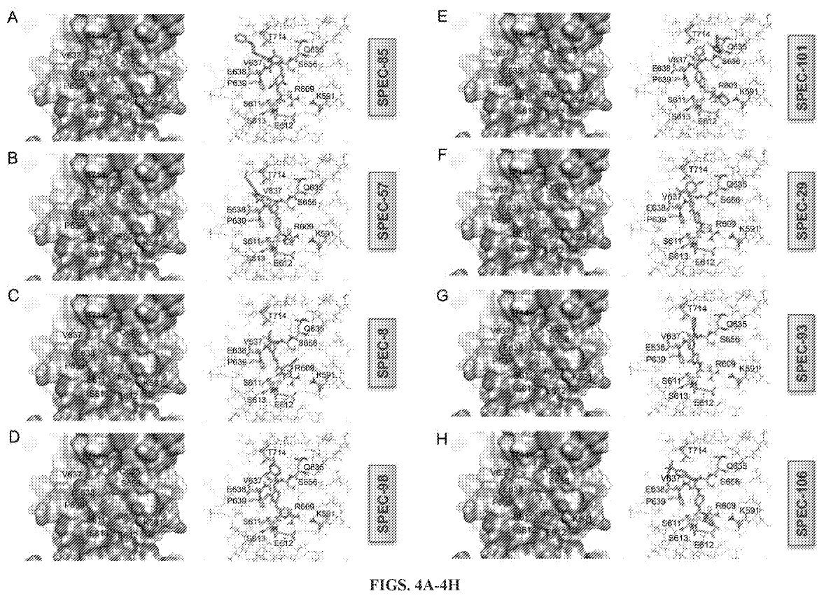

Binding mode of CJ-887 with STAT3. CJ-887 is a potent peptidomimetic inhibitor of STAT3 with a Ki value of 15 nM (Chen et al., 2010). It was designed by modifying the phosphorylated hexapeptide (pY₉₀₅LPQTV) derived from gp130 protein residues 905-910 (Gomez et al., 2009). CJ-887 competitively binds to the SH2 domain of STAT3 and inhibits STAT3 homodimerization, a pre-requisite for its high-affinity binding to duplex DNA (Gomez et al., 2009). The binding mode of CJ-887 revealed by our docking experiments showed that it is similar to the binding orientation of the STAT3 pY705 peptide (AAPpY₇₀₅LKTKFICVTPF) as assessed from the crystal structure of the STAT3-dimer (Becker et al., 1998). The native pY705 peptide-binding site includes an “U” shaped area of interface with that surrounds the projection formed by the side chain of E638 (FIGS. 2A & 2B). Three sub-pockets are defined within this area of interface: 1) the pY+0 pocket that binds pY705, 2) the pY+1 pocket that binds L706, and 3) a hydrophobic side pocket and that binds pY-X [FIG. 2; Park and Li, 2011]. The pY705 residue forms a hydrogen bonds with side chains of R609 and S613 in the pY+0 pocket. The phosphorylated phenol in CJ-887, which serves as a pY705 mimic, (2D structure shown in FIG. 3A) binds to the pY+0 pocket (FIG. 2C) by forming hydrogen bonds with the side chain of R609 within the pocket and K591 within αA, one of the four alpha helices described in the crystal structure of STAT30 (Becker et al., 1998). The amide side chain in CJ-887 is located on the opposite side of E638, in relation to the phospho-phenol group and forms a hydrogen bond with the carbonyl group of the peptide backbone of E638. Interestingly, the amide side chain of Gln residues within peptide-based STAT3 inhibitors (Coleman et al., 2005) also bind the SH2 domain at this position, which is of critical importance for their binding affinity.

Peptide immunoblot affinity assays and mirror resonance affinity analysis of phosphopeptides derived from growth factor receptors, e.g. EGFR peptide pY₁₀₆₈XXQ, also demonstrated that only pY-peptides containing Gln at the +3 position (not Leu, Met, Glu, or Arg) bound to STAT3, through H-bonds between the oxygen within the +3 Gln side chain and the backbone amide of Glu-638 (Shao et al., 2004). As expected, removal of the amide side chain from CJ-887 resulted in a significant loss of potency, indicating that hydrogen bonds formed here also are key interactions for its binding (Coleman et al., 2005). The bicyclic lactam ring located inside the pY+1 pocket and made contact with surrounding residues, including L706. Moreover, the carbonyl moiety from the bicyclic lactam formed an additional hydrogen bond with the amide group from the backbone of E638 (FIG. 2C). Thus, three groups of hydrogen bonds formed by different parts of CJ-887, anchored it within the “U” conformation of STAT3 (FIG. 2C), mimicking the interactions of different fragments of the phosphorylated peptide (FIG. 2B).

Binding site flexibility. To relax the docking pose, the complex structure of CJ-887 and STAT3 SH2 domain was subjected to 10 ns molecular dynamic (MD) simulations to allow for conformational adjustment of both ligand (CJ-887) and protein (STAT3). The averaged structure from the last 2 ns MD simulation was extracted and is shown in FIG. 3. CJ-887 in the averaged structure still anchors within the “U” shape interface around the projection of the E638 sidechain. The hydrogen bond formed by the bicyclic lactam and the backbone carbonyl of E638 remained unchanged after the simulation. Additionally, the side chain of Q644 flips toward the amide group of CJ-887 and forms a new hydrogen bond replacing the hydrogen bond formed with the backbone of E638 in the docking pose. The hydrogen bond network in pY+0 pocket also changed after the simulation. CJ-887 is tightly hydrogen bonded with the side chain hydroxyl group and backbone amide group from S613 while the hydrogen bonds between the phosphorylated phenol and R609 and K591 disappear (FIG. 3C). By comparing the protein structures before and after MD simulation, it was found that αA helix where K591 is located, conducts movements outward from the central R-sheet strand, causing a change in the spatial distance between the phosphate group and amino side chain of K591 (FIG. 3C, FIG. 11A). The movements of the αA and K591 are critical in that, they introduce a larger space in pY+0 pocket in the averaged structure (FIG. 11B & 11C). In contrast, the positions of conserved residues R609-S613 and V637-P639 remained unchanged.

The orientation of phospho-phenol moiety of CJ-887 has been adjusted to position it parallel with the flat “wall” formed by side chain and backbone atoms of E638 and P639, allowing hydrophobic contacts in this area (FIG. 3B). In addition to hydrogen bonding, the bicyclic ring also contacts with the hydrophobic side chain of V637 and T714 (FIG. 3B). The relative position of the main chain of CJ-887 did not change significantly before or after MD simulation due to the aforementioned hydrogen networks and hydrophobic interactions. Distinct from the main chain of CJ-887, the side chains including the phenyl ring and the acetamide moiety flip away from the original location. No strong polar or hydrophobic interactions are identified between the acetamide group and STAT3, indicating it contributes little to protein binding (FIG. 3C, FIG. 11A). Indeed, the acetamide site was chosen to introduce long lipid chains to increase the cellular permeability for derivatives of CJ-887 without significant influence to binding affinity (Chen et al., 2010). The simulation results are consistent with this experimental data.

In silico screening and STAT3 inhibitory properties of the hits. The averaged structure derived from MD simulation was used as the receptor for ligand docking and high throughput virtual screening (HTVS) to evaluate binding affinities of 110,000 compounds in the SPEC database. After re-docking and re-scoring thirty percent of the top-ranking molecules with standard precision (SP) parameter settings, the top 10% were selected as inputs into the pose filter along with further restrictions. Importantly, the pose filter was defined to select only the poses that formed hydrogen bonds with residues R609 and S614 in the pY+0 pocket. Many of the top-ranking poses did not form hydrogen bonds with these residues and were filtered out. Subsequently, 110 compounds, fulfilling these criteria, were purchased to test for their ability to inhibit granulocyte colony-stimulating factor (G-CSF)-induced phosphotyrosylation of STAT3 (pY-STAT3) in Kasumi-1 cells, as described (Redell et al., 2011). Twenty-four compounds inhibited G-CSF-induced pY-STAT3 by more than 50% at a concentration of 10 μM with 9 compounds at this concentration inhibiting pY-STAT3 by more than 99% (Supplemental Table 1).

SUPPLEMENTAL TABLE 1
Results of screening of 110 SPEC compounds for ability
to inhibit G-CSF-stimulated pSTAT3 activation in
Kasumi1 cells, at a single concentration of 10 μM

| | | % of Control |
|---|---|---|
| LAB | SPEC | Stimulated |
| FILE# | IDNUMBER | pSTAT3 |
| 8 | AP-355/42609662 | 0 |
| 93 | AG-690/37048015 | 0 |
| 29 | AN-979/41971071 | 0 |
| 32 | AN-806/14212005 | 0 |
| 57 | AN-023/41981716 | 1 |
| 106 | AF-399/15284578 | 1 |
| 21 | AN-989/41696610 | 1 |
| 109 | AE-641/01209003 | 1 |
| 101 | AG-205/36715027 | 1 |
| 85 | AH-487/41138477 | 2 |
| 90 | AG-690/40752406 | 4 |
| 86 | AH-487/40936629 | 5 |
| 98 | AG-690/09291009 | 20 |
| 82 | AH-487/41661411 | 27 |
| 20 | AN-989/41696613 | 27 |
| 1 | AQ-380/42570523 | 28 |
| 65 | AK-968/41924598 | 35 |
| 73 | AK-918/43446771 | 38 |
| 102 | AG-205/36494060 | 38 |
| 100 | AG-205/37106187 | 42 |
| 103 | AG-205/13057008 | 46 |
| 87 | AK-968/40379914 | 47 |
| 72 | AK-968/12971227 | 47 |
| 66 | AK-968/40732022 | 48 |
| 92 | AG-690/40700249 | 51 |
| 34 | AN-698/41888408 | 53 |
| 28 | AN-989/14669143 | 53 |
| 75 | AK-918/43337065 | 55 |
| 97 | AG-690/11453692 | 55 |
| 25 | AN-989/40748327 | 58 |
| 27 | AN-989/14177017 | 61 |
| 83 | AH-487/41659679 | 61 |
| 18 | AQ-022/43452377 | 66 |
| 99 | AG-670/15094030 | 67 |
| 89 | AG-690/40756163 | 68 |
| 30 | AN-919/40736828 | 68 |
| 7 | AP-427/40954608 | 68 |
| 35 | AN-648/41914111 | 69 |
| 2 | AQ-360/42670517 | 70 |
| 39 | AN-648/41885151 | 70 |
| 53 | AN-329/15332069 | 71 |
| 46 | AN-465/14458037 | 71 |
| 41 | AN-648/40682114 | 74 |
| 21 | AG-690/40700265 | 74 |
| 12 | AO-081/40926778 | 75 |
| 40 | AN-648/41665123 | 75 |
| 3 | AQ-088/42014341 | 77 |
| 37 | AN-648/41888938 | 77 |
| 59 | AM-879/37189004 | 78 |
| 61 | AH-487/41955121 | 78 |
| 79 | AH-487/42145419 | 79 |
| 110 | AA-788/33245021 | 80 |
| 38 | AN-648/41885166 | 80 |
| 4 | AQ-088/42013878 | 81 |
| 6 | AP-853/43368098 | 82 |
| 26 | AG-690/15437974 | 82 |
| 74 | AK-918/43446327 | 82 |
| 11 | AO-081/41227729 | 86 |
| 105 | AF-399/36013003 | 86 |
| 51 | AN-329/15538278 | 87 |
| 10 | AO-375/15014027 | 87 |
| 54 | AH-487/41143012 | 87 |
| 6 | AP-906/41637264 | 89 |
| 33 | AN-698/42227391 | 89 |
| 80 | AH-487/42145031 | 89 |
| 104 | AG-205/12075024 | 91 |
| 14 | AO-080/43441688 | 91 |
| 9 | AO-375/41189759 | 93 |
| 50 | AN-329/40388457 | 94 |
| 58 | AH-034/11365498 | 95 |
| 16 | AO-050/43342627 | 96 |
| 22 | AN-989/41696408 | 96 |
| 43 | AN-465/43411023 | 96 |
| 94 | AG-690/15442569 | 97 |
| 19 | AN-989/41898728 | 97 |
| 60 | AM-879/15327266 | 98 |
| 38 | AN-648/41667104 | 98 |
| 15 | AO-080/43378493 | 98 |
| 108 | AE-848/32325009 | 98 |
| 13 | AO-081/15245115 | 99 |
| 31 | AN-919/14229175 | 99 |
| 70 | AK-988/15363549 | 99 |
| 64 | AK-988/41924833 | 100 |
| 58 | AM-900/15050059 | 101 |
| 68 | AK-968/15609871 | 101 |
| 54 | AN-329/14789049 | 102 |
| 95 | AG-690/15441581 | 104 |
| 47 | AN-329/41402668 | 104 |
| 24 | AN-989/41695997 | 105 |
| 49 | AN-329/40543516 | 105 |
| 78 | AK-918/42829291 | 105 |
| 17 | AO-048/13641016 | 106 |
| 71 | AK-968/15380845 | 106 |
| 107 | AE-848/32742054 | 107 |
| 87 | AH-262/34336021 | 107 |
| 62 | AM-807/41628553 | 108 |
| 58 | AN-329/12715748 | 110 |
| 48 | AN-329/40723556 | 112 |
| 69 | AK-968/15604564 | 112 |
| 45 | AN-465/41674806 | 113 |
| 61 | AM-879/14885007 | 114 |
| 55 | AN-329/14085035 | 114 |
| 23 | AN-989/41696352 | 114 |
| 78 | AI-204/31681043 | 116 |
| 28 | AN-988/40650028 | 117 |
| 42 | AN-465/43411318 | 118 |
| 63 | AK-968/41925208 | 118 |
| 52 | AN-329/15538276 | 120 |
| 44 | AN-465/43410995 | 120 |
| 77 | AK-918/42829287 | 121 |

Note:
Kasumi1 cells were pre-treated with inhibitor (10 μM) and then stimulated with G-CSF (100 ng/ml, [symbol] ), media removed, cells washed with cold P [symbol] DS and pellets used to prepare protein which was tested for pSTAT3, GAPDH [symbol] . % of control was calculated by dividing pSTAT3/GAPDH of inhibitor treated cells by [symbol] of DMSO treated cells. expressed as %. [symbol]
indicates data missing or illegible when filed

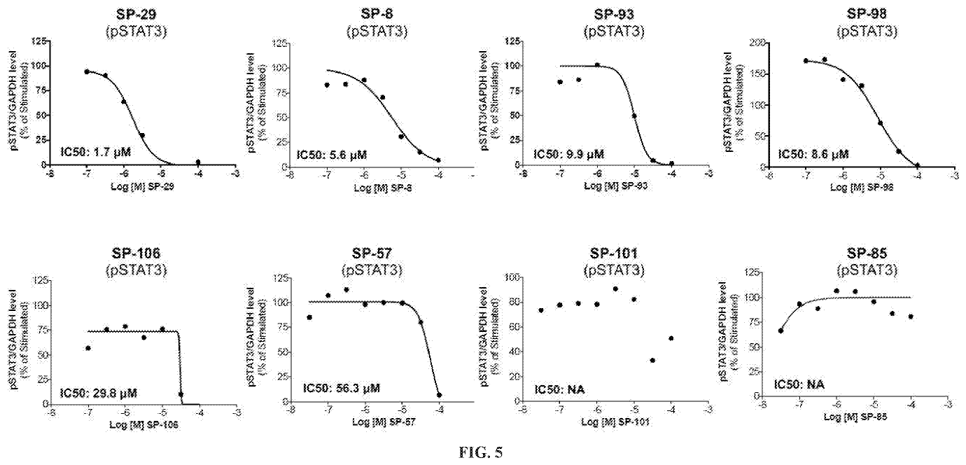

Surface Plasmon Resonance (SPR) was performed on the 24 compounds to determine their ability to block binding of purified STAT3 to an immobilized phosphododecapeptide based on EGFR Y1068 (EGFR pY-peptide), as described (Xu et al., 2009). Eight compounds (SPEC-29, 8, 93, 98, 106, 57, 101 and 85; see Table A) that inhibited G-CSF-stimulated pY-STAT3 by 20-80% inhibition (Supplemental Table 1) inhibited STAT3 binding to EGFR pY-peptide by 29% to 71% at 10 μM and by 67% to 93% at 100 μM (Supplemental Table 2). The binding poses of the 8 compounds, shown in FIG. 4, demonstrate two common features-occupation of the pY+0 pocket and formation of hydrogen bonds with R609 or S611-S614 per the molecular selection criteria. These 8 compounds were then evaluated for IC₅₀ of inhibition of G-CSF-stimulated pY-STAT3 in Kasumi-1 cells. Five compounds (SPEC-29, 8, 93, 98, and 106) had appreciable inhibitory activity with IC₅₀s ranging from 2.7-19.0 μM (Table 1, FIG. 5). SPEC29 and 8 were identified as most potent compounds and the IC₅₀ s of 2.7 and 4.1 μM, respectively.

TABLE 1
Identification numbers and inhibitory activities of SPEC compounds

| | | | Growth AI | | Growth AD | |
|---|---|---|---|---|---|---|
| | | G-CSF pSTAT3 | IC50 (μM) | | IC50 (μM) | |
| Comp # | SPEC ID | IC50 (μM) | 468 | 231 | 468 | 231 |
| SPEC-29 | AN-979/41971071 | 2.7 ± 1.4 | 1.8 ± 1.6 | 9.5 ± 4.1 | 2.4 ± 2.5 | 3.0 ± 2.2 |
| SPEC-8 | AP-355/42609662 | 4.1 ± 2.2 | 6.6 ± 4.1 | 26.1 ± 1.3 | 6.3 ± 4.6 | 5.5 ± 3.0 |
| SPEC-93 | AG-690/37048015 | 10.4 ± 0.8 | 11.1 ± 4.5 | 15.7 ± 1.3 | 12.1 ± 5.4 | 12.2 ± 0.3 |
| SPEC-98 | AG-690/09291009 | 14.2 ± 8.1 | 64.1 ± 3.1 | NA | 32.5 ± 3.7 | 25.6 ± 3.9 |
| SPEC-106 | AF-399/15284578 | 19.0 ± 12.7 | 34.9 ± 1.3 | 38.9 | 44.7 ± 0.0 | 39.0 ± 0.0 |
| SPEC-57 | AN-023/41981716 | 34.5 ± 30.4 | 77.5 ± 47.1 | NA | 44.0 ± 26.9 | 65.2 ± 17.3 |
| SPEC-101 | AG-205/36715027 | 99.0 ± 43.8 | 20.4 ± 1.0 | 48.6 ± 30.8 | 26.9 ± 0.1 | 21.0 ± 2.4 |
| SPEC-85 | AH-487/41138477 | >100 | NA | NA | NA | NA |

Note:
Comp # is Lab ID provided and has been used to refer to a compound all though the manuscript.
SPEC ID is the ID provided at SPECS database at http://www.specs.net
Abbreviations:
NA: No Activity: NA:
AD: Anchorage dependent,
AI: anchorage independent
Cells used: Kasumi1 for G-CSF induced pSTAT3/GAPDH by Luminex, 468: MDA-MB-468, 231; MDA-MB-231

| LAB | | % of max | % of max |
|---|---|---|---|
| FILE | | binding | binding |
| # | SPEC IDNUMBER | at 10 μm | at 100 μm |
| 8 | AP-355/42809662 | 49.1 | 30.3 |
| 93 | AG-690/37048015 | 116.8 | 28.3 |
| 29 | AN-979/41971071 | 69.6 | 22.1 |
| 32 | AN-808/14212005 | 82.8 | 44.3 |
| 57 | AN-023/41981718 | 28.7 | 33.5 |
| 106 | AF-399/15284578 | 68.9 | 20.4 |
| 21 | AN-989/41698610 | 71.0 | 46.1 |
| 109 | AE-641/01209003 | 97.1 | 82.3 |
| 101 | AG-205/36715027 | 72.9 | 9.4 |
| 85 | AH-487/41138477 | 67.3 | 9.5 |
| 90 | AG-690/40752406 | 132.2 | 59.5 |
| 86 | AH-487/40935629 | 120.1 | 59.1 |
| 98 | AG-890/09291009 | 70.4 | 7.8 |
| 82 | AH-487/41681411 | 109.3 | 28.4 |
| 20 | AN-989/41696613 | 76.7 | 46.1 |
| 1 | AQ-360/42570523 | 80.0 | 44.7 |
| 65 | AK-968/41924598 | 57.5 | 43.5 |
| 73 | AK-918/43446771 | 58.5 | 31.5 |
| 102 | AG-205/36494060 | 73.7 | 21.4 |
| 100 | AG-205/37108187 | 70.8 | 15.4 |
| 103 | AG-205/13057008 | 76.0 | 10.4 |
| 67 | AK-968/40379914 | 64.1 | 51.3 |
| 72 | AK-968/12971227 | 56.8 | 40.6 |
| 66 | AK-968/40732022 | 57.2 | 15.9 |

Note:
pEGFRp-STAT3 binding (SPR) inhibition at pH 8. By selected SPEC compounds was done as described in method.

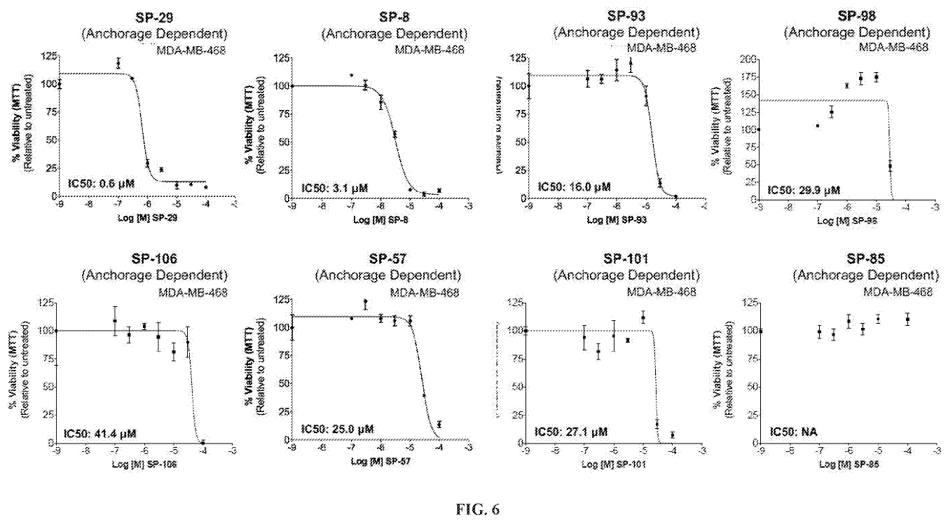

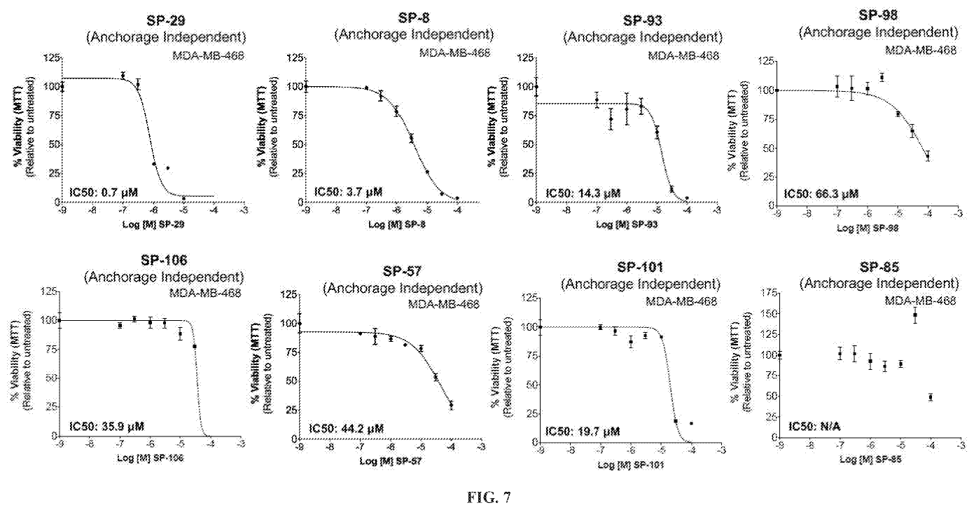

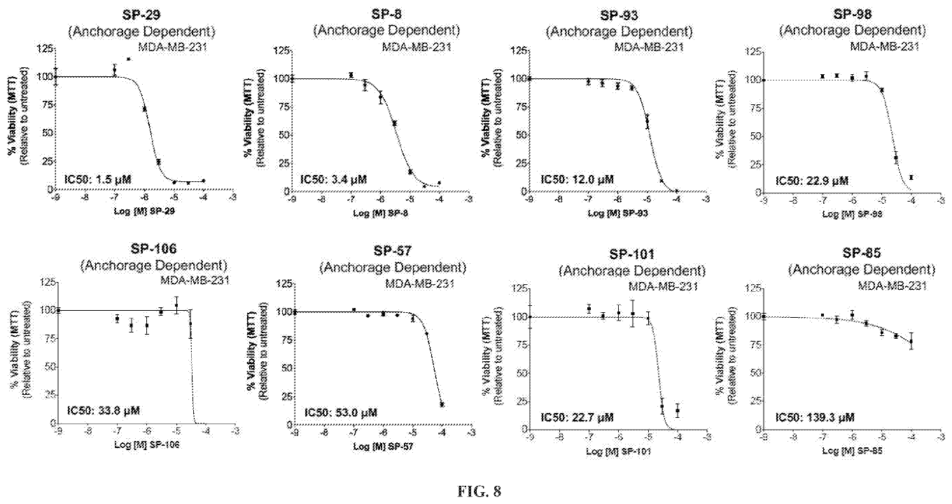

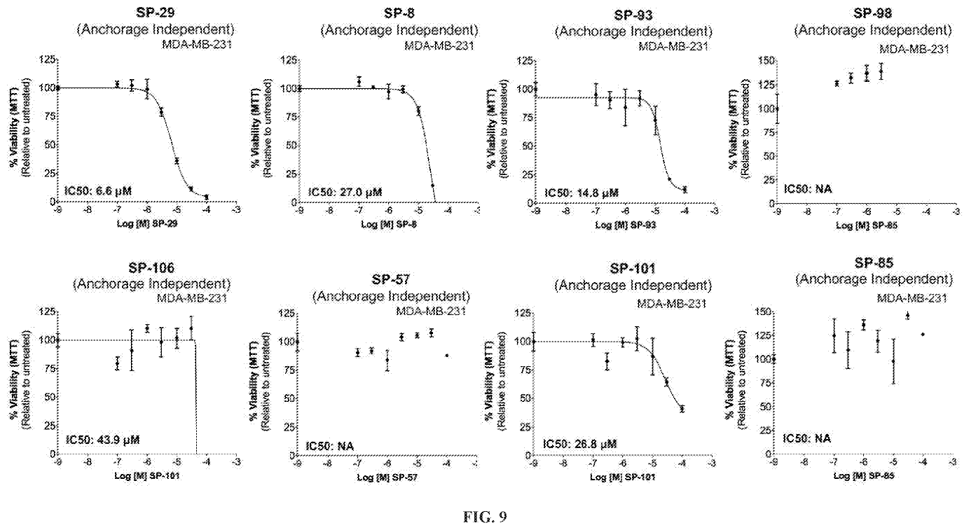

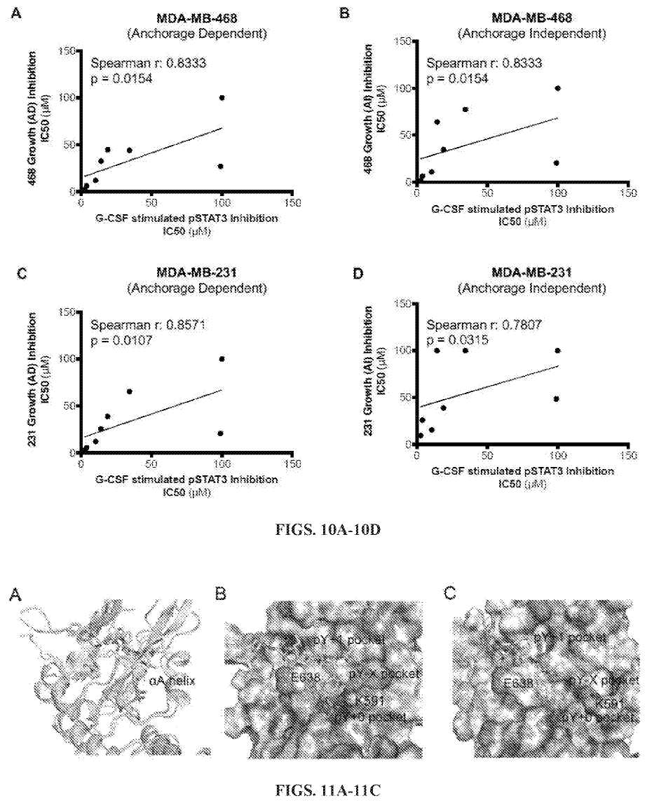

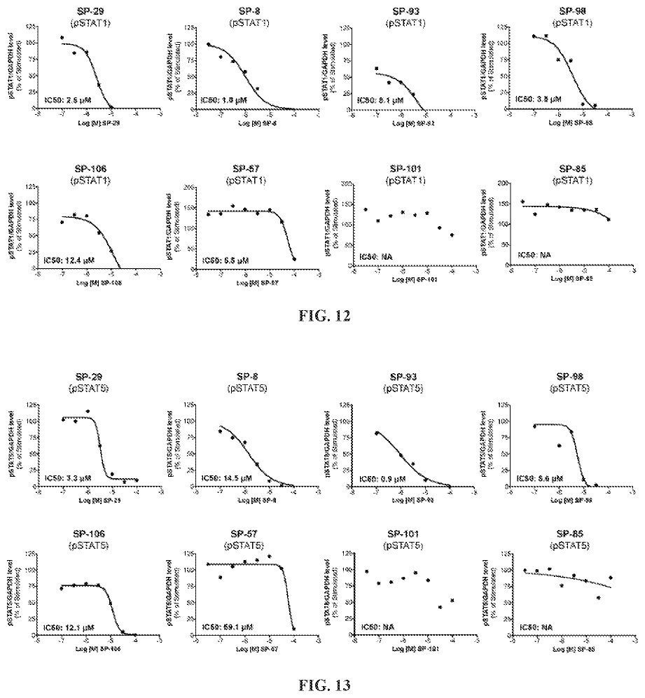

Evaluation of cell growth inhibition by SPEC compounds. To evaluate compounds for their anti-cancer properties, we measured their ability to inhibit growth of two breast cancer lines, MDA-MB-468 and MDA-MB-231, known to express increased levels of pY-STAT3 (Marotta et al., 2011) as to depend on STAT3 for their survival (Marotta et al., 2011), drug resistance (Tan et al., 2015), and metastatic ability (Thakur et al., 2015). SPEC-29, 8, and 93 (Table 1) potently inhibit MDA-MB-468 cell growth under conditions of anchorage dependent conditions (AD, IC₅₀=2.4-12.1 μM; FIG. 6), as well as anchorage independent conditions (AI, IC₅₀ s=1.8-11.1 μM; FIG. 7). Similar results were obtained for MDA-MB-231 (AD IC₅₀ s=3.0-12.2 μM; FIG. 8). Inhibition of anchorage independent growth of MDA-MB-231 was similar to that for MDA-MB-468, except that SPEC-8 showed an unexpectedly high IC₅₀ (26.1 μM; FIG. 9). The remaining five compounds showed less potency in inhibiting growth of these cell lines (AD and IC IC₅₀ ˜20-80 μM) with SPEC-85 showing no activity against either cell line. The ability of the eight compounds to inhibit growth of MDA-MB-468 cells, correlated positively with their abilities to inhibit G-CSF-stimulated pSTAT3 levels (AD: Spearman r=0.8333, p=0.015, FIG. 10A; AI: Spearman r=0.8333, p=0.015, FIG. 10B), as well as MDA-MB-231 (AD: Spearman r=0.8571, p=0.0107, FIG. 10C; AI: Spearman r=0.7807, p=0.0315, FIG. 10D). These results strongly suggest that the ability of these SPEC compounds to inhibit growth of pY-STAT3-dependent cells depends on their ability to reduced levels of pY-STAT3 in these cells.

### D. Discussion

Virtual ligand screening of the SPECS chemical library was performed for the first time using a structure derived from MD simulation of the STAT3 SH2 domain in complex with a high-affinity ligand (CJ-887). The screen yielded 110 compounds as potential STAT3 inhibitors. Eight initial hits were identified by pSTAT3 (G-CSF-stimulated)-inhibitory and STAT3-pEGFRp binding inhibitory (SPR)-screening assays (Table A and Table 1). IC₅₀ determination experiments for hit validation clearly revealed at least six compounds (SPEC-29/8/93/98 and 106) with appreciable pSTAT3 inhibitory activities ranging from 2.7-19.0 μM. Three compounds (SPEC-29/8/93) with lowest IC50s for G-CSF-stimulated pY-STAT3 inhibition (≤10 μM), were also the most potent in inhibiting pSTAT3-driven growth of breast cancer lines (IC₅₀: 2.4-12.2 μM, anchorage dependent). The remaining three compounds, SPEC-98, 106 and 57, showed poor cell growth inhibitory activity (IC₅₀: 25.6-65.2 μM, anchorage dependent). The abilities of the compounds to inhibit growth of pSTAT3-driven breast cancer cell lines correlated to their abilities to inhibit G-CSF)-stimulated pY-STAT3 activity (FIG. 10). Drug-like properties of these compounds were also evaluated to determine their suitability to develop into effective STAT3-directed, anti-oncogenic drugs (Table 2). Considering all the activities, the screening process yielded at least two highly potent STAT3 inhibitors, SPEC-29 and 8, of which SPEC-29 seems to be the most promising.

The drug-likeness of the eight initial hits (2D chemical structures shown in Table A) was assessed using Lipinski's four “rules of five” (Lipinski et al., 2001). Seven out of eight compounds comply with three or more of the four rules (Table 2), with the first four compounds (SPEC-29, 8, 93, and 98) fulfilling all 4 rules. Clearly, SPEC29 and SPEC-8, with most potent anti-pSTAT3 and cell growth inhibitory activities, both fulfilled all four rules, indicating their likeliness for being candidate small molecule anti-STAT3 drugs.

Table 2
Molecular and pharmacological characteristics of the eight hit compounds.

| | | | | | Hbond | Hbond | Rotatable |
|---|---|---|---|---|---|---|---|
| Comp # | Chemical name | SPEC ID | Mol Wt | LogP | acceptors | donors | bonds |
| SPEC-29 | N-(4-[(E)-3-(5-bromo-2-methoxyphenyl)prop-2- | AN-979/41971071 | 374.23 | 3.74 | 3 | 1 | 6 |
| | enoyl)phenyl]acetamide | | | | | | |
| SPEC-8 | 5-bromo-2-hydroxy-3-[3-(4-methoxyphenyl)acryloyl]- | AP-355/42609662 | 361.19 | 2.76 | 3 | 1 | 4 |
| | 2,4,6-cycloheptatrien-1-one | | | | | | |
| SPEC-93 | N-(2-aniline-4′-methyl-4,5′-bi-1,3-thiazol-2′-yl)acetamide | AG-690/37048015 | 330.43 | 4.21 | 5 | 2 | 5 |
| SPEC-98 | 5[4-[2,4-dichlorobenzyl)oxy]-3-methoxybenzylidene]-1,3- | AG-690/09291009 | 410.28 | 4.39 | 3 | 1 | 5 |
| | thiazolidine-2,4-diene | | | | | | |
| SPEC-106 | N-(4-{[(2,6-dimethyl-4-pyrimidinyl)amino]sulfonyl}phenyl)- | AF-399/15284578 | 530.65 | 5.82 | 7 | 2 | 10 |
| | 2-[4-(1-methyl-1-phenylethyl)phenoxy]acetamide | | | | | | |
| SPEC-57 | 4-(5-([1-(4-ethylphenyl)-3,5-dioxo-4-pyrazol8dinyldene]meth- | AN-023/41981716 | 520.59 | 3.86 | 8 | 2 | 7 |
| | yl}-2-furyl)-N-(1,3-thiazol-2-yl)benzenesulfonamide | | | | | | |
| SPEC-101 | N-(2{[2-(4-morpholinyl)-2-oxoethyl]sulfanyl}-1,3-benzo- | AG-205/36715027 | 563.69 | 2.59 | 9 | 0 | 9 |
| | thiazol-6-yl)-N-({5-[(1-phenyl-1H-tetraazol-5-yl)sulfonyl]-2- | | | | | | |
| | furyl}methylene)amine | | | | | | |
| SPEC-85 | [2,6-dibromo-4-({2,4-dioxo-3-[2-oxo-2-(4-phenyl-1-piperazin- | AH-487/41138477 | 639.32 | 4.17 | 8 | 1 | 8 |
| | yl)ethyl]-1,3-thiazolidin-5-ylidene}methyl)phenoxy]acetic acid | | | | | | |

Note:
Lipinski's ‘rule of 5’ predicts that poor absorption or permeation is more likely when there are more than 5 H-bond donors. 10 H-bond acceptors, the molecular weight (MWT) is greater than 500 and the calculated octanol-water partition coefficient. Log P (CLogP) is greater than 5 (or MlogP > 4.15)

The ranking of the final eight compounds based on docking results using crystal structure as receptor and standard precision parameter set (Ranking SP1) as well as docking using the averaged structure from MD simulation as the receptor (Ranking SP2) are shown in Table 3. It is clearly evident that the binding site flexibility remarkably affects the ranking of compounds in virtual screening. Most of the eight final hits were poorly scored or ranked in the docking experiment based on crystal structure, e.g. SPEC-85 was ranked among the top 30% (33,000) from crystal structure HTVS screening (HTVS1) and ordered as 5201 in the SP1 screening. The two others that were ranked high in SP1 were SPEC-93 (954) and SPEC-106 (1007). The other compounds were not ranked within the top 10,000, in the crystal structure SP screening. However, in the two-step averaged structure screening (SP2), six compounds (SPEC-85/57/8/93/98 and 29) were ranked within top 1,000 while two (SPEC-106/101) ranked within top 5,000.

TABLE 3
Docking results of eight SPEC compounds using crystal structure or averaged structure from MD simulation

| | | Ranking | glide gscore | Ranking | glide gscore | Ranking | glide gscore | Ranking | glide gscore |
|---|---|---|---|---|---|---|---|---|---|
| Comp # | SPEC ID | HTVS1 | HTVS1 | SP1 | SP1 | HTVS2 | HTVS2 | SP2 | SP2 |
| SPEC-85 | AH-487/41138477 | 13209 | −4.38 | 5201 | −5.16 | 7545 | −4.63 | 525 | −5.89 |
| SPEC-57 | AN-023/41981716 | 21598 | −4.17 | | — | 16827 | −4.31 | 596 | −5.86 |
| SPEC-8 | AP-355/42609662 | — | — | | — | 686 | −5.37 | 611 | −5.85 |
| SPEC-93 | AG-690/37048015 | 1020 | −5.31 | 954 | −5.76 | 8039 | −4.60 | 639 | −5.84 |
| SPEC-98 | AG-690/09291009 | — | — | | — | 626 | −5.39 | 821 | −5.76 |
| SPEC-29 | AN-979/41971071 | — | — | | — | 542 | −5.43 | 977 | −5.71 |
| SPEC-106 | AP-399/15284578 | 3240 | −4.94 | 1007 | −5.75 | 18420 | −4.26 | 3880 | −5.28 |
| SPEC-101 | AG-205/36715027 | 11558 | −4.44 | | — | 4072 | −4.84 | 4697 | −5.21 |

Notes:
HTVS1: docking experiment using crystal structure as the receptor and High Throughput Virtual Screening parameter set.
SP1: docking experiment using the crystal structure as the receptor and Standard Precision parameter set
HTVS2: docking experiment using the averaged structure from MD simulation as the receptor and High Throughput Virtual Screening parameter set
SP2: docking experiment using the averaged structure from MD simulation as the receptor andStandard Precision parameter set.
Compounds are arranged as per their rank according to SP2.
Some of the compounds could not be docked to the binding pocket. e.g. no reasonable binding pose could be found by the docking procedure. Since we ranked the compounds by docking score, and these compounds, don't have a docking score, they did not show up in the ranking list.

The binding pattern analysis of the docking experiments using averaged structure derived from MD simulation revealed that, although the 2D chemical structures (Table A) are diverse for the eight hits, they occupied similar binding sites on SH2 domain (FIG. 4). Screening criterion (described in methods and FIG. 1) ensured that all hits exhibited hydrogen-bonding interactions with S611-S613 in the pY+0 pocket area (FIG. 4, Table 4) although each had different chemical moieties. Phosphate or phosphorylated phenol group occupied the pY+0 pocket in the poses of the crystal structure of SH2 dimer (FIG. 2, Table 4) and docking model of CJ-887 (FIG. 2C, FIG. 3, and Table 4) respectively. Most of the known STAT3 inhibitors also harbor negatively charged moieties to mimic p-peptide interactions in this area (Debnath et al., 2012; and Xu et al., 2009). Interestingly, negatively charged carboxyl group in SPEC-85 forms hydrogen-bonding interactions in pY+0 pocket; while for the other seven compounds, neutral charge groups are located in this area (Table 4). Thus, seven out of the eight compounds (except SPEC-85) identified as hits are electrically neutral at physiological pH suggesting that charged groups are not necessary to bind the pY+0 pocket STAT3 in our model. Based on this finding, one may hypothesize that many potentially strong inhibitors may have been overlooked in earlier studies, based on the classical modeling using STAT3 static crystal structure (Bharadwaj et al., 2016). Due to their inherent bias for a negative charge at the pY+0 pocket, these studies might have, in essence, looked for only charged compounds unlike the hits we found, which have polar atoms, instead of negatively charged groups, forming hydrogen-bonding interactions. This is especially important, as, charged molecules such as CJ-887 suffer from poor cell membrane permeability and, thus, are far less suitable for clinical development. In fact, this has been a main reason that inhibitors targeting SH2-domains of many other targets e.g. Src kinase, the Src-family kinase Lck, p85, the regulatory subunit of PI3K and Grb2 have also been generally unsuccessful (Morlacchi et al., 2014). The phosphotyrosine (pY) residue was estimated to provide one half of the binding energy of phosphopeptides to the SH2 domain (Bradshaw et al., 1999; Bradshaw and Waksman, 1999; and Grucza et al., 1999) and hence considered an absolute necessity. At one point in time, therefore, the idea of targeting a SH2 domain was virtually abandoned (Morlacchi et al., 2014). The d neutral compounds identified in this study (e.g. SPEC-29/8), are thus, good candidates for STAT3 hit-to-lead drug development and a similar strategy might be successful in designing inhibitors targeting SH2 domains within other oncogenic targets as well.

TABLE 4
Hydrogen bonds formed between STAT3 dimer, CJ-887 and active
SPEC compounds in the pY + 0 pocket.

| | Chemical moieties | Residues forming | Charge |
|---|---|---|---|
| | forming hydrogen | hydrogen bonds in | of the |
| Entity | bonds in Py + 0 pocket | Py + 0 pocket | moiety |
| STAT3 dimer | —O—PO3 | E612-NH | −ve |
| | | S613-OH | |
| CJ-887 | —O—PO3 | S613-NH | −ve |
| | | S613-OH | |
| SPEC-85 | —COO | E612-NH | −ve |
| | | S613-NH | |
| SPEC-57 | —SO2— | E612-NH | neutral |
| | | S613-NH | |
| SPEC-8 | 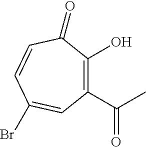 | R609-NH2 E612-NH | neutral |
| SPEC-96 | 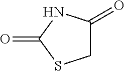 | E612-NH S613-NH | neutral |
| SPEC-101 | 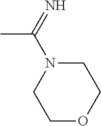 | E612-NH K591-NH3 | neutral |
| SPEC-29 | 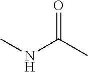 | E612-NH S613-NH | neutral |
| SPEC-93 | 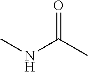 | E612-NH S613-NH | neutral |
| SPEC-106 | —SO2— | E612-NH | neutral |
| | | S613-NH | |

One of the reasons for large polar groups not being able to bind to the pY+0 pocket might be the relatively narrow size of the pY+0 pocket in the crystal structure, which is incapable of accommodating large groups with potential to form hydrogen-bonds with S611-5613 (FIGS. 11B & 11C). For instance, SPEC-8, and SPEC-98 are not able to dock to the crystal structure due to the limited space, whereas both of the compounds were reordered within top 1,000 list based on the average structure (Table 3). The relatively large chemical moieties, trienone group from SPEC-8 and thiazolidine group in SPEC-98, positioned at the bottom of pY+0 pocket according to the docking poses derived from the STAT3 averaged structure (FIGS. 4C & 4D, Table 4). It seems that the movement of αA helix and side chain of K591, in the induced-fit model resulted in a larger pY+0 pocket, and hence better ranking for compounds harboring big chemical moieties (FIG. 11). The strategy of incorporating the MD simulation to accommodate the SH2 domain flexibility, thus, tends to uncover new classes of compounds never identified before.

In addition to the hydrogen bonding at the pY+0 pocket, another common feature is the hydrophobic interactions between the aromatic or hydrophobic groups in the compounds and flat “wall” formed by side chain and backbone atoms of V637, E638 and P639, which are also observed in the docking pose of CJ-887 (FIG. 3). For SPEC-85 and SPEC-57, a hydrogen bond was predicted to form between the carbonyl group within compounds and the amide group from backbone of E638 (FIGS. 4A & 4B). This hydrogen bond is also observed in the docking pose of CJ-887 and maintained during the MD simulation. But the high IC₅₀ (G-CSF-pSTAT3) of these two compounds (SPEC85/57 specially SPEC85), proved that the contribution of this interaction might not be as important as the pY-interactions. SPEC-8 is the smallest inhibitor with the best ligand efficiency of binding energy per atom as calculated (Kuntz et al., 1999) and represents the smallest set of interactions necessary for potency, both the hydrogen-bonding network in pY+0 pocket and hydrophobic interactions with the flat wall (FIG. 4C). The IC₅₀-pSTAT3 of the compound is also one of the lowest (4.0 μM).

SPEC-29 is the hit with second-lowest molecular weight. The binding pose of SPEC-29 (FIG. 4F) is typical, with its acetamide group forming hydrogen bonds in pY+0 pocket, phenyl ring forming hydrophobic interactions with E638 flat wall and 1-bromo-4-methoxybenzene group locating near the T714, making contacts with hydrophobic pY+1 pocket. The G-CSF-pSTAT3-IC₅₀ of this one is the lowest (2.7 μM). The hydrogen bonds with S611 and S613, the hydrophobic contacts with pY+0 and pY+1 pocket, and the relative low rotatable bonds may contribute to its high in vitro efficacies.

Following the initial in-silico predicted binding of 110 SPEC compounds, we tested 24/110 hits, that suppressed STAT3 phosphorylation by more than 50% at 10 μM (Supplemental Table 1) for ability to inhibit binding of pEGFR-peptide to recombinant STAT3 in SPR assays (at 10 and 100 μM, Supplemental Table 2). Eight compounds that showed appreciable activity in both assays were selected for further analysis. The IC₅₀ of for inhibition of G-CSF-stimulated pSTAT3 by these eight compounds however (Table 1) did not correlate with their ability to inhibit pEGFRp-STAT3 binding (Supplemental Table 2). The lack of correlation was especially striking for two compounds—SPEC-85, which showed 95% reduction in pEGFRp-binding of STAT3 by SPR at 100 μM (Supplemental Table 2) while having almost no activity against G-CSF-pSTAT3 (Table 1) and SPEC-29, which showed 78% inhibition of peptide binding at 100 μM by SPR assay (Supplemental Table 2), while being the most potent inhibitor of G-CSF-pSTAT3 with a mean IC₅₀ of 2.7 μM (Table 1).

The discordance between biochemical and cellular activity of SPEC-85 could result from its presumably low permeability due to its negative charge, as well as its big size (MW 639, Table 2). In fact, the IC50s for G-CSF-pSTAT3 inhibition by the eight compounds, correlated with their molecular weights (Pearson R=0.7768, p=0.0234, Spearman R=0.8743, p=0.0079), indicating the importance of smaller size on the intracellular activity of the compounds. Many previous studies also have shown that cellular activity (IC₅₀) of STAT3 inhibitors generally did not linearly correlate with thermodynamically deciphered Kds (Park and Li, 2011; Dhanik et al., 2011; and Dhanik et al., 2012).

Molecular dynamic studies using parameters obtained from binding studies of p-peptide (and/or STAT3) to STAT3 (Lin et al., 2099; and Poli et al., 2016), peptidomimetics to STAT3 (Dhanik et al., 2011; and Dhanik et al., 2012) or small molecules to STAT3 (Park and Li, 2011; and Shao et al., 2014) have been performed previously. However, the present study may be the first to use a ligand-STAT3 bound-fit model to screen libraries by docking. The data presented herein indicate the strength of this approach for identifying hits with a neutral moiety that binds the pY+0 pocket and may prove to be extremely useful in further hit-to-lead development.

All of the compounds, compositions, and/or methods disclosed and claimed herein can be made and executed without undue experimentation in light of the present disclosure. While the compounds, compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference:
Anderson, Practical Process Research & Development—A Guide for Organic Chemists, 2^{nd} ed., Academic Press, New York, 2012.
Baker et al., “Electrostatics of nanosystems: application to microtubules and the ribosome”, Proc Natl Acad Sci USA, 98(18):10037-10041, 2001.
Becker et al., “Three-dimensional structure of the Stat3beta homodimer bound to DNA”, Nature, 394(6689):145-151, 1998.
Bharadwaj et al., “Drug-repositioning screening identified piperlongumine as a direct STAT3 inhibitor with potent activity against breast cancer”, Oncogene, 34(11):1341-1353, 2015.
Bharadwaj et al., “Small-Molecule Inhibition of STAT3 in Radioresistant Head and Neck Squamous Cell Carcinoma”, Oncotarget, 7(18):26307-26330, 2016a.
Bharadwaj et al., “STAT3 Inhibitors in Cancer: A Comprehensive Update”, In: Ward A C, editor. STAT Inhibitors in Cancer. Cham: Springer International Publishing, pp 95-161, 2016b.
Bradshaw and Waksman, “Calorimetric examination of high-affinity Src SH2 domain-tyrosyl phosphopeptide binding: dissection of the phosphopeptide sequence specificity and coupling energetics”, Biochemistry, 38(16):5147-5154, 1999.
Bradshaw et al., “Investigation of phosphotyrosine recognition by the SH2 domain of the Src kinase”, J Mol Biol, 293(4):971-985, 1999.
Bromberg and Darnell, “The role of STATs in transcriptional control and their impact on cellular function”, Oncogene, 19(21):2468-2473, 2000.
Bromberg et al., “Stat3 as an Oncogene”, Cell, 98(3):295-303, 1999.
Case et al., AMBER 11 User's Manual. University of California, San Francisco, 2010.
Chen et al., “Structure-Based Design of Conformationally Constrained, Cell-Permeable STAT3 Inhibitors”, ACS Med Chem Lett, 1(2):85-89, 2010.
Cieplak et al., “Application of the multimolecule and multiconformational RESP methodology to biopolymers: Charge derivation for DNA, RNA, and proteins”, J Comp Chem, 16(11):1357-1377, 1995.
Coleman et al., “Investigation of the binding determinants of phosphopeptides targeted to the SRC homology 2 domain of the signal transducer and activator of transcription 3. Development of a high-affinity peptide inhibitor”, J Med Chem, 48(21):6661-6670, 2005.
Darden “Particle mesh Ewald: An N [center-dot] log(N) method for Ewald sums in large systems”, J Chem Phys, 98(12):10089-10092, 1993.
Darnell, “STATs and Gene Regulation”, Science, 277(5332):1630-1635, 1997.
Darnell, “Validating Stat3 in cancer therapy”, Nat Med, 11(6):595-596, 2005.
Debnath et al., “Small molecule inhibitors of signal transducer and activator of transcription 3 (Stat3) protein”, J Med Chem, 55(15):6645-6668, 2012.
Dhanik et al., “Binding modes of peptidomimetics designed to inhibit STAT3”, PLoS One, 7(12):e51603, 2012.
Dhanik et al., “On modeling peptidomimetics in complex with the SH2 domain of Stat3”, Conf Proc IEEE Eng Med Biol Soc, 2011:3229-3232, 2011.
Diao and Meibohm, “Pharmacokinetics and pharmacokinetic-pharmacodynamic correlations of therapeutic peptides”, Clin Pharmacokinet, 52(10):855-868, 2013.
Dupradeau et al., “The R.E.D. tools: advances in RESP and ESP charge derivation and force field library building”, Phys Chem Chem Phys, 12(28):7821-7839, 2010.
Egloff and Grandis, “Improving Response Rates to EGFR-Targeted Therapies for Head and Neck Squamous Cell Carcinoma: Candidate Predictive Biomarkers and Combination Treatment with Src Inhibitors”, J Oncol, 2009:896407, 2009.
Germain and Frank, “Targeting the cytoplasmic and nuclear functions of signal transducers and activators of transcription 3 for cancer therapy”, Clin Cancer Res, 13(19):5665-5669, 2007.
Glide v. Schrodinger, LIC.: New York, N.Y., 2011.
Gomez et al., “Design, synthesis, and evaluation of peptidomimetics containing Freidinger lactams as STAT3 inhibitors”, Bioorg Med Chem Lett, 19(6):1733-1736, 2009.
Grucza et al., “SH2 domains: from structure to energetics, a dual approach to the study of structure-function relationships”, Med Res Rev, 19(4):273-293, 1999.
Handbook of Pharmaceutical Salts: Properties, and Use, Stahl and Wermuth Eds., Verlag Helvetica Chimica Acta, 2002.
Hocker et al., “Andrographolide derivatives inhibit guanine nucleotide exchange and abrogate oncogenic Ras function”, Proc Natl Acad Sci USA, 110(25):10201-10206, 2013.
Hornak et al., “Comparison of multiple Amber force fields and development of improved protein backbone parameters”, Proteins, 65(3):712-725, 2006.
Hsieh et al., “Evaluation of potential Stat3-regulated genes in human breast cancer”, Biochem Biophys Res Comm, 335(2):292-299, 2005.
Izaguirre et al., “Langevin stabilization of molecular dynamics”, J Chem Phys, 114(5):2090-2098, 2001.
Jiang et al., “Peptidomimetic inhibitors of APC-Asef interaction block colorectal cancer migration”, Nat Chem Biol, 13(9):994-1001, 2017.
Jing and Tweardy, “Targeting Stat3 in cancer therapy”, Anti-Cancer Drugs, 16(6):601-607, 2005.
Jorgensen et al., “Comparison of simple potential functions for simulating liquid water”, J Chem Phys, 79(2):926-935, 1983.
Kuntz et al., “The maximal affinity of ligands”, Proc Natl Acad Sci USA, 96(18):9997-10002, 1999.
Leeman et al., “STAT3 as a therapeutic target in head and neck cancer”, Expert Opin Biol Ther, 6(3):231-241, 2006.
Leeman-Neill et al., “Honokiol inhibits epidermal growth factor receptor signaling and enhances the antitumor effects of epidermal growth factor receptor inhibitors”, Clin Cancer Res, 16(9):2571-2579, 2010.
Levy and Darnell, “STATs: transcriptional control and biological impact”, Nat Rev Mol Cell Biol, 3(9):651-662, 2002.
Lieblein et al., “STAT3 can be activated through paracrine signaling in breast epithelial cells”, BMC Cancer, 8(1):302, 2008.
LigPrep v. Schrodinger, LIC.: New York, N.Y., 2011.
Lin et al., “Molecular dynamics simulations of the conformational changes in signal transducers and activators of transcription, Stat1 and Stat3”, J Mol Graph Model, 28(4):347-356, 2009.
Lipinski et al., “Experimental and computational approaches to estimate solubility and permeability in drug discovery and development settings”, Adv Drug Deliv Rev, 46(1-3):3-26, 2001.
Maestro v. Schrodinger, LIC.: New York, N.Y., 2011.
Marotta et al., “The JAK2/STAT3 signaling pathway is required for growth of CD44CD24 stem cell-like breast cancer cells in human tumors”, J Clin Invest, 121(7):2723-2735, 2011.
Marotta et al., “The JAK2/STAT3 signaling pathway is required for growth of CD44+CD24− stem cell-like breast cancer cells in human tumors”, J Clin Invest, 121(7):2723-2735, 2011.
Morlacchi et al., “Targeting SH2 domains in breast cancer”, Future Med Chem, 6(17):1909-1926, 2014.
Park and Li, “Characterization of molecular recognition of STAT3 SH2 domain inhibitors through molecular simulation”, J Mol Recognition, 24(2):254-265, 2011.
Poli et al., “Identification of a new STAT3 dimerization inhibitor through a pharmacophore-based virtual screening approach”, J Enzyme Inhib Med Chem, 31(6):1011-1017, 2016.
Reagan-Shaw et al., FASEB J., 22(3):659-661, 2008.
Redell et al., “Stat3 signaling in acute myeloid leukemia: ligand-dependent and -independent activation and induction of apoptosis by a novel small-molecule Stat3 inhibitor”, Blood, 117(21):5701-5709, 2011.
Ren et al., “Identification of a high-affinity phosphopeptide inhibitor of Stat3”, Bioorg Med Chem Lett, 13(4):633-636, 2003.
Ryckaert et al., “Numerical integration of the cartesian equations of motion of a system with constraints: molecular dynamics of n-alkanes”, J Comp Phys, 23(3):327-341, 1977.
Shao et al., “Dual-inhibitors of STAT5 and STAT3: studies from molecular docking and molecular dynamics simulations”, J Mol Model, 20(8):2399, 2014.
Shao et al., “Structural requirements for signal transducer and activator of transcription 3 binding to phosphotyrosine ligands containing the YXXQ motif”, J Biol Chem, 279(18):18967-18973, 2004.
Shen et al., “ASD v3.0: unraveling allosteric regulation with structural mechanisms and biological networks”, Nucleic Acids Res, 44(D1):D527-535, 2016.
Shen et al., “Proteome-Scale Investigation of Protein Allosteric Regulation Perturbed by Somatic Mutations in 7,000 Cancer Genomes”, Am J Hum Genet, 100(1):5-20, 2017.
Siddiquee et al., “An Oxazole-Based Small-Molecule Stat3 Inhibitor Modulates Stat3 Stability and Processing and Induces Antitumor Cell Effects”, ACS Chem Biol, 2(12):787-798, 2007.
Siddiquee et al., “Selective chemical probe inhibitor of Stat3, identified through structure-based virtual screening, induces antitumor activity”, Proc Natl Acad Sci, 104(18):7391-7396, 2007.
Smith, March's Advanced Organic Chemistry: Reactions, Mechanisms, and Structure, 7^{th} Ed., Wiley, 2013.
Song et al., “A low-molecular-weight compound discovered through virtual database screening inhibits Stat3 function in breast cancer cells”, Proc Natl Acad Sci, 102(13):4700-4705, 2005.
Tan et al., “Src/STAT3-dependent heme oxygenase-1 induction mediates chemoresistance of breast cancer cells to doxorubicin by promoting autophagy”, Cancer Sci, 106(8):1023-1032, 2015.
Thakur et al., “Inhibition of STAT3, FAK and Src mediated signaling reduces cancer stem cell load, tumorigenic potential and metastasis in breast cancer”, Sci Rep, 5:10194, 2015.
Turkson et al., “Novel peptidomimetic inhibitors of signal transducer and activator of transcription 3 dimerization and biological activity”, Mol Cancer Ther, 3(3):261-269, 2004.
Turkson et al., “Phosphotyrosyl peptides block Stat3-mediated DNA binding activity, gene regulation, and cell transformation”, J Biol Chem, 276(48):45443-45455, 2001.
Vinkemeier et al., “DNA binding of in vitro activated Stat1 alpha, Stat1 beta and truncated Stat1: interaction between NH2-terminal domains stabilizes binding of two dimers to tandem DNA sites”, EMBO J, 15(20):5616-5626, 1996.
Wang et al., “Development and testing of a general amber force field”, J Comput Chem, 25(9):1157-1174, 2004.
Wen et al., “Maximal activation of transcription by stat1 and stat3 requires both tyrosine and serine phosphorylation”, Cell, 82(2):241-250, 1995.
Xu et al., “Chemical Probes that Competitively and Selectively Inhibit Stat3 Activation”, PLoS One, 4(3):e4783, 2009.
Xu et al., “Cooperative DNA binding and sequence-selective recognition conferred by the STAT amino-terminal domain”, Science, 273(5276):794-797, 1996.
Zhang et al., “Therapeutic effects of STAT3 decoy oligodeoxynucleotide on human lung cancer in xenograft mice”, BMC Cancer, 7:149, 2007.

1. A pharmaceutical composition comprising:
(A) a compound of the formula: 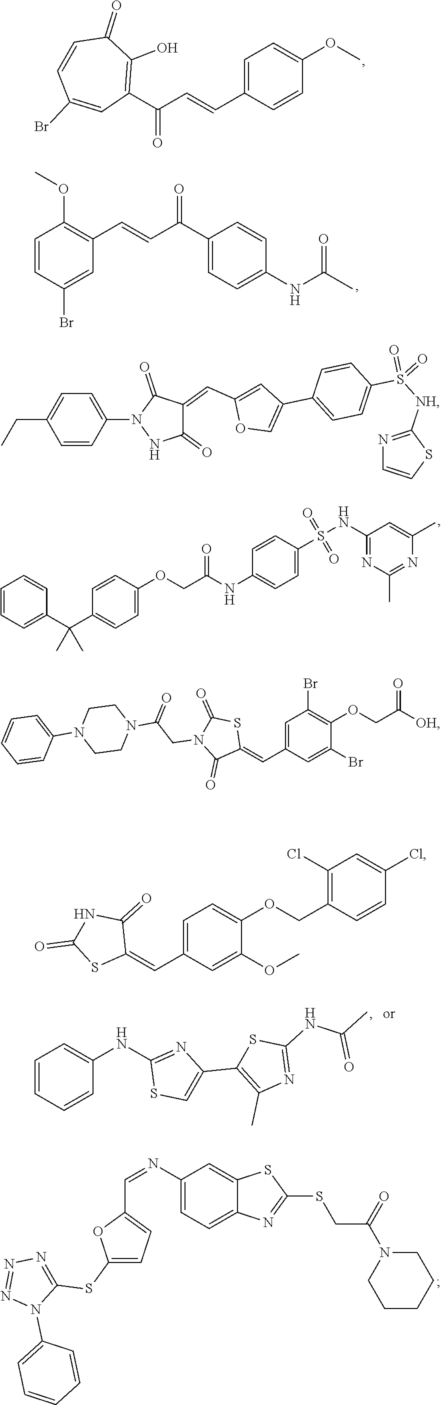
or a pharmaceutically acceptable salt thereof; and
(B) an excipient.

2.-7. (canceled)

8. A method of inhibiting STAT3 in a cell comprising contacting the cell with an effective amount of a compound of the formula: 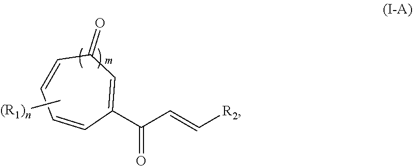
wherein:
n is 0, 1, 2, or 3;
m is 0 or 1;
R₁ is, in each instance independently, hydrogen, halo, hydroxy, amino, cyano, or nitro; or
alkyl_{(C≤6)}, alkylamino_{(C≤6)}, dialkylamino_{(C≤6)}, alkoxy_{(C≤6)}, acyloxy_{(C≤6)}, amido_{(C≤6)}, or a substituted version of any of these groups; and
R₂ is aryl_{(C≤8)}, substituted aryl_{(C≤8)}, heteroaryl_{(C≤8)}, or substituted heteroaryl_{(C≤8)};
provided the compound is not: 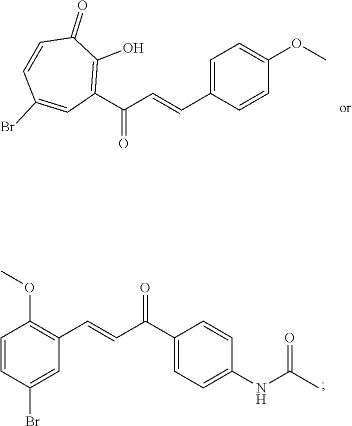 [SMILES: COc1ccc(/C=C/C(=O)c2cc(Br)ccc(=O)c2O)cc1 ; COc1ccc(Br)cc1/C=C/C(=O)c1ccc(NC(C)=O)cc1]
a compound of the formula: 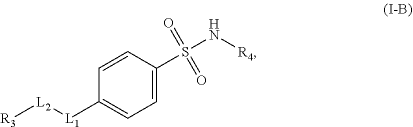
wherein:
L₁ is arenediyl_{(C≤8)}, heteroarenediyl_{(C≤8)}, —NHC(O)-alkanediyl_{(C≤6)}-O—, or a substituted version of any of these groups;
L₂ is arenediyl_{(C≤8)} or substituted arenediyl_{(C≤8)}; or
a group of the formula: 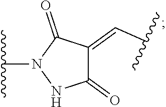 [SMILES: */C=C1\C(=O)NN(*)C1=O]
R₃ is aryl_{(C≤8)}, aralkyl_{(C≤8)}, heteroaryl_{(C≤8)}, heteroaralkyl_{(C≤8)}, or a substituted version of any of these groups; and
R₄ is aryl_{(C≤8)}, substituted aryl_{(C≤8)}, heteroaryl_{(C≤8)}, or substituted heteroaryl_{(C≤8)};
provided the compound is not: 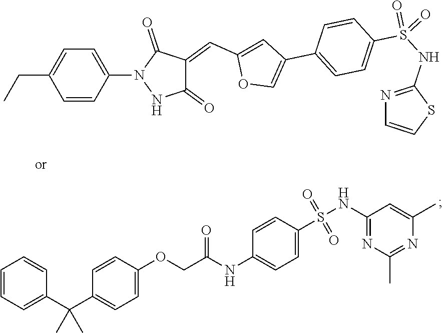 [SMILES: CCc1ccc(N2NC(=O)/C(=C\c3cc(-c4ccc(S(=O)(=O)Nc5nccs5)cc4)co3)C2=O)cc1 ; Cc1cc(NS(=O)(=O)c2ccc(NC(=O)COc3ccc(C(C)(C)c4ccccc4)cc3)cc2)nc(C)n1]
or
a compound of the formula: 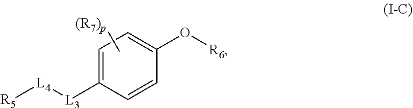
wherein:
p is 0, 1, or 2;
L₃ is a group of the formula: 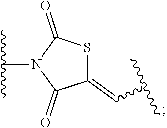
L₄ is a covalent bond, -heterocycloalkanediyl_{(C≤8)}-C(O)-alkanediyl_{(C≤6)}-, or substituted -heterocycloalkanediyl_{(C≤8)}-C(O)-alkanediyl_{(C≤6)}-;
R₅ is hydrogen, aryl_{(C≤8)}, substituted aryl_{(C≤8)}, heteroaryl_{(C≤8)}, or substituted heteroaryl_{(C≤8)};
R₆ is alkyl_{(C≤6)}, substituted alkyl_{(C≤8)}, aralkyl_{(C≤6)}, or substituted aralkyl_{(C≤8)}; and
R₇ is, in each instance independently, hydrogen, halo, hydroxy, amino, cyano, or nitro; or
alkyl_{(C≤6)}, alkylamino_{(C≤6)}, dialkylamino_{(C≤6)}, alkoxy_{(C≤6)}, acyloxy_{(C≤6)}, amido_{(C≤6)}, or a substituted version of any of these groups;
provided that the compound is not: 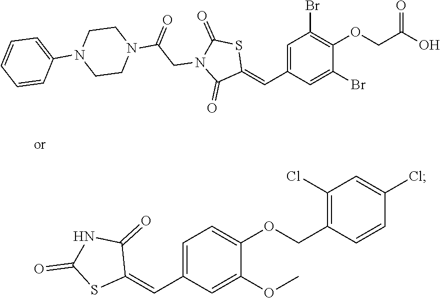 [SMILES: O=C(O)COc1c(Br)cc(/C=C2\SC(=O)N(CC(=O)N3CCN(c4ccccc4)CC3)C2=O)cc1Br ; COc1cc(/C=C2/SC(=O)NC2=O)ccc1OCc1ccc(Cl)cc1Cl]
or
a compound of the formula: 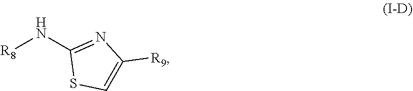
wherein:
R₈ and R₉ are each independently aryl_{(C≤8)}, substituted aryl_{(C≤8)}, heteroaryl_{(C≤8)}, or substituted heteroaryl_{(C≤8)};
provided the compound is not: 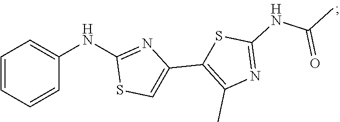 [SMILES: CC(=O)Nc1nc(C)c(-c2csc(Nc3ccccc3)n2)s1]
a compound of the formula: 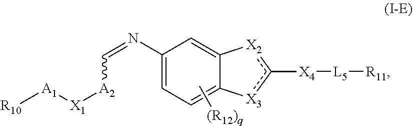
wherein:
q is 0, 1, 2, or 3;
A₁ and A₂ are each independently arenediyl_{(C≤8)}, substituted arenediyl_{(C≤8)}, heteroarenediyl_{(C≤8)}, or substituted heteroarenediyl_{(C≤8)};
L₅ is a covalent bond, —C(O)—, -alkanediyl_{(C≤6)}-C(O)—, or substituted -alkanediyl_{(C≤6)}-C(O)—;
R₁₀ is alkyl_{(C≤6)}, aryl_{(C≤8)}, aralkyl_{(C≤8)}, or substituted version of any of these groups;
R₁₁ is alkyl_{(C≤6)}, cycloalkyl_{(C≤8)}, heterocycloalkyl_{(C≤8)}, or substituted version of any of these groups;
R₁₂ is, in each instance independently, hydrogen, halo, hydroxy, amino, cyano, or nitro; or
X₁ and X₄ are each independently —O—, —S—, or —NRₐ—, wherein:
Rₐ is hydrogen, alkyl_{(C≤6)}, or substituted alkyl_{(C≤6)}; and
X₂ and X₃ are each independently —O—, —S—, —N═, —NR_{b}—, wherein:
R_{b} is hydrogen, alkyl_{(C≤6)}, or substituted alkyl_{(C≤6)};
provided that one of X₂ or X₃ is not —N═ and provided the compound is not: 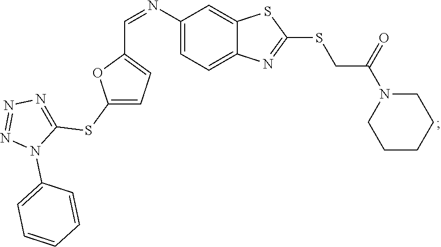 [SMILES: O=C(CSc1nc2ccc(/N=C\c3ccc(Sc4nnnn4-c4ccccc4)o3)cc2s1)N1CCCCC1]
or a pharmaceutically acceptable salt of these formulae.

9.-21. (canceled)

22. The method of claim 8, wherein R₁ is bromo or acetamido and R₂ is 4-methoxyphenyl or 5-bromo-2-methoxyphenyl.

23.-30. (canceled)

31. The method of claim 8, wherein L₁ is furan-3,5-diyl or —NHC(O)CH₂O—and L₂ is benzen-1,4-diyl or a group of the formula: 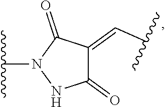 [SMILES: */C=C1\C(=O)NN(*)C1=O]
R₃ is 4-ethylphen-1-yl or 1,1-dimethyl-1-phenylmethyl, and R₄ is thiazol-2-yl or 2,6-dimethylpyrimidin-4-yl.

32.-50. (canceled)

51. The method of claim 8, wherein L₄ is a covalent bond or -piperazin-1,4-diyl-C(O)CH₂—, R₅ is hydrogen or phenyl, R₆ is carboxymethyl or 2,4-dichlorophenyl, and R₇ is bromo or methoxy.

52.-71. (canceled)

72. The method of claim 8, wherein R₈ is phenyl and R₉ is 2-acetamido-4-methylthiazol-5-yl.

73.-81. (canceled)

82. The method of claim 8, wherein X₁, X₂, and/or X₄ is —S—; X₃ is —N═; A1 is tetrazol-1,5-diyl: A₂ is furan-2,5-diyl: L₅ is —CH₂C(O)—; R₁₀ is phenyl; and R₁₁ is N-piperidinyl.

83.-95. (canceled)

96. A method of inhibiting STAT3 in a cell comprising contacting the cell with an effective amount of a compound of the formula: 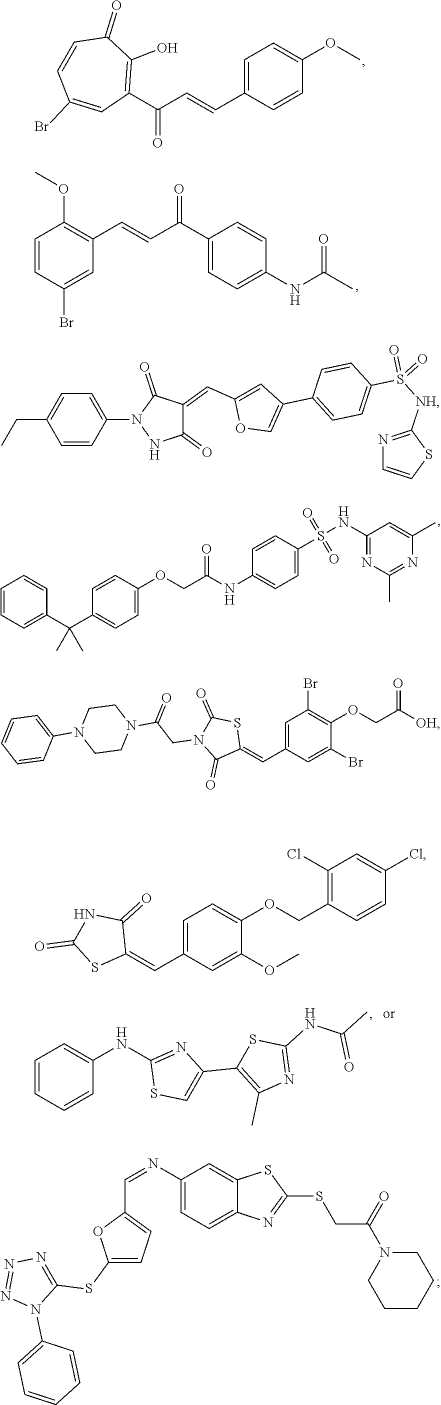
or a pharmaceutically acceptable salt thereof.

97. The method of claim 96, wherein the cell is an immune cell or a cancer cell.

98. (canceled)

99. The method of claim 96, further defined as a method of treating a subject and comprising administering an effective amount of comprising the compound to the subject.

100. The method of claim 99, wherein the subject has an autoimmune disease, an inflammatory disease, or a cancer.

101. The method of claim 100, wherein the inflammatory disease is atherosclerosis, peripheral vascular disease, coronary artery disease, hypertension, osteoporosis, type 2 diabetes, or dementia.

102. The method of claim 100, wherein the subject has a cancer.

103. The method of claim 102, wherein the cancer is a metastatic cancer.

104. The method of claim 102, wherein the cancer overexpresses STAT3 or exhibits increased STAT3 activation.

105. The method of claim 102, wherein the cancer is a breast cancer.

106. The method of claim 102, wherein the cancer is a carcinoma or a hematological cancer.

107. The method of claim 106, wherein the cancer is leukemia.

108. The method of claim 107, wherein the cancer is acute myeloid leukemia (AML).

109. The method of claim 102, further comprising administering an immune check point inhibitor therapy to the subject.

110.-111. (canceled)
